(19) United States
(12) Patent Application Publication
(10) Pub. No.: US 2022/0265635 A1
(43) Pub. Date: Aug. 25, 2022

(54) TREATMENT OF NEUROLOGICAL DISEASE

(71) Applicants: Aclipse One Inc., Radnor, PA (US); The University of Sheffield, Sheffield (GB)

(72) Inventors: Richard James Mead, Sheffield (GB); Pamela Jean Shaw, Derbyshire (GB); Claude Ogoe, Monrovia, CA (US); Ning Shan, Chandler, AZ (US); Laura Ferraiuolo, Sheffield (GB)

(21) Appl. No.: 17/286,797

(22) PCT Filed: Oct. 18, 2019
(86) PCT No.: PCT/US19/56996

§ 371 Date: Apr. 19, 2021

Related U.S. Application Data
(60) Provisional application No. 62/747,961, filed on Oct. 19, 2018.

(51) Int. Cl. A61K 31/473; A61P 25/28

(52) U.S. Cl. CPC A61K 31/473; A61P 25/28

(57) ABSTRACT

The invention is directed to 6-methyl-5,6,6a,7-tetrahydro-4H-dibenzo[de,g]quinoline-10,11-diol for the treatment of diseases mediated by protein misfolding, heat shock factor 1 pathways, or nuclear erythroid 2-related factor 2 pathways.

### CROSS-REFERENCE TO RELATED APPLICATION

The present application claims the benefit of U.S. Provisional Application No. 62/747,961, filed Oct. 19, 2018, the disclosure of which is hereby incorporated by reference in its entirety.

### FIELD OF INVENTION

The present invention relates to a therapeutic agent and methods for the treatment of diseases mediated by protein misfolding, the Heat Shock Protein Factor 1 (HSF1) pathway, or nuclear erythroid 2—related factor 2 (NRF2) pathway.

### BACKGROUND

In normal cells, protein homeostasis is maintained by regulating the expression, folding, modification, translocation and, ultimately, degradation of proteins. To achieve this, cells use sophisticated mechanisms to ensure the proper execution of these processes in response to cellular stress. A crucial aspect of cellular protein homeostasis is the utilization of chaperone proteins, which stabilize protein structures, assist in correct folding and unfolding of proteins, and facilitate the assembly of multimeric protein complexes. Chaperone proteins, including αB-crystallin, heat shock protein 27 (HSP27), HSP40, HSP70 and HSP90, as well as class I and class II chaperonins, function individually or as part of larger heterocomplexes to prevent protein misfolding, the accumulation of misfolded proteins, and protein aggregation. Chaperone proteins can promote cell survival by stabilizing and refolding misfolded proteins, and by inhibiting apoptosis.

Heat shock transcription factor 1 (HSF1, HGNC:5224 https://www.ncbi.nlm.nih.gov/gene/3297) is the master activator of chaperone protein gene expression. HSF1 promotes the expression of genes encoding chaperone proteins in response to cellular stress. It also regulates the expression of genes involved in other aspects of cell survival, including protein degradation, ion transport, signal transduction, energy generation, carbohydrate metabolism, vesicular transport and cytoskeleton formation.

Stress-dependent regulation of HSF1 is a multistep process that is controlled by intricate regulatory mechanisms. Under basal conditions, HSF1 exists largely as an inactive monomer in the cytoplasm, repressed in part through the activity of the chaperone proteins HSP90, HSP70, HSP40, the chaperonin containing t-complex polypeptide 1 (TCP1) ring complex (TRiC), and other co-chaperones which form an inhibitory complex with HSF1. In response to proteotoxic stress, HSF1 is thought to dissociate from HSP90, HSP70, HSP40, and TRiC. This allows HSF1 to homotrimerize, accumulate in the nucleus, and, after activating post-translational modifications (including phosphorylation), bind to heat shock elements in the promoter region of target stress-responsive genes, including those encoding chaperone proteins.

Protein chaperones play critical roles in protein synthesis, de novo folding, refolding, disaggregation, oligomeric assembly, trafficking, modification, maturation and degradation, either via Ubiquitin Proteasome System and/or autophagy (including Chaperone Mediated Autophagy) of cellular client proteins. The HSF1 pathway has been implicated in a diverse range of diseases including cancer, neurodegenerative disease, metabolic diseases, inflammatory disease and cardiovascular disease, involving a range of cells and tissues including neuronal, heart, muscle, spleen and liver.

Protein misfolding, accumulation of misfolded proteins, and protein aggregation are the hallmarks of neurodegenerative diseases. In patients with Parkinson's disease, Parkinson's disease dementia, or dementia with Lewy bodies, Lewy bodies are observed in the cytoplasm of neurons of the substantia nigra in the brain. The major constituents of these aggregates are fragments of a protein named α-synuclein, phosphorylated α-synuclein, hyperphosphorylated Tau, Leucine-Rich Repeat Kinase 2 (LRRK2), and trans-active DNA binding protein 43 (TDP-43) aggregates. In Alzheimer's disease, there are mainly 2 types of protein deposits. Amyloid plaques are deposited extracellularly in the brain parenchyma and around the cerebral vessel walls, and their main component is a 40- to 42-residue peptide termed β-amyloid protein. Neurofibrillary tangles are located in the cytoplasm of degenerating neurons and are composed of aggregates of hyperphosphorylated tau protein. Up to 50% of Alzheimer's disease patients are also known to have TDP-43 aggregates in the central nervous system (CNS). In patients with Huntington disease, intranuclear deposits of an expanded polyglutamine version of mutated huntingtin protein is a typical feature of the brain. Patients with amyotrophic lateral sclerosis (ALS) have misfolded and/or aggregated proteins mostly TDP-43 (either mutant or misregulated), and/or other proteins including misfolded ubiquitinated aggregates TDP43, the protein encoded by the TAR DNA binding protein 43, and misfolded superoxide dismutase (SOD1), dipeptide repeat proteins from expanded hexanucleotide repeat C9orf72, hyphosphorylated Tau, Fused in Sarcoma (FUS), Ataxin-2 (ATXN2), and heterogenous nuclear ribonucleoproteins (hnRNPs) in cell bodies and axons of motor neurons and/or interneurons. Finally, the brains of humans and animals with diverse forms of transmissible spongiform encephalopathy are characterized by accumulation of protease-resistant aggregates of the prion protein.

The pharmacological activation of HSF1 and the HSF1 pathway is a promising avenue for therapeutic intervention in diseases involving the HSF1 pathway and, in particular, those involving protein misfolding, accumulation of misfolded proteins, and protein aggregation. HSF1 activators represents a novel therapeutic strategy that could slow, halt, or reverse the underlying disease process in diseases involving the HSF1 pathway.

One group of diseases that can be treated by therapeutic interventions involving HSF1 pathway are mitochondrial diseases. Mitochondrial diseases are genetic disorders that are driven by genetic mutations in mitochondrial DNA (mtDNA) and nuclear DNA (nDNA) of genes that are transcribed and translated into mitochondrial proteins that are responsible for mitochondrial function. These mutations result in the misfolding and aggregation of mutated mitochondrial proteins/enzymes which impair mitochondrial function including oxidative phosphorylation, fatty acid oxidation, Krebs cycle, urea cycle, gluconeogenesis and ketogenesis. (Gorman et al. Nat Rev Dis Primers. 2016, 2, 1-22).

HSF1 activation has been shown to restore impaired mitochondrial proteostasis and improve mitochondrial function, remove terminally dysfunctional mitochondria via mitophagy, and regenerate new mitochondria via mitochondrial biogenesis. These improvements in mitochondrial function and biogenesis leads to increased oxidative phosphorylation, thermogenesis and energy expenditure. (Gomez-Pastor et al. Nat Rev Mol Cell Biol. 2018, 19, 4-19).

Diseases/disorders due to mitochondrial dysfunction as a result of genetic mutation include:

Childhood-Onset mitochondrial diseases—Leigh syndrome; Alpers-Huttenlocher syndrome; Childhood myocerebrohepatopathy spectrum (MCHS); Ataxia neuropathy spectrum (ANS); Myoclonic epilepsy myopathy sensory ataxia (MEMSA); Sengers syndrome; MEGDEL syndrome; Pearson syndrome; and Congenital lactic acidosis (CLA); Adult-Onset mitochondrial diseases—Leber hereditary optic neuropathy (LHON); Kearns-Sayre syndrome (KSS); Mitochondrial myopathy, encephalopathy, lactic acidosis and stroke-like episodes (MELAS) syndrome; Myoclonic epilepsy with ragged red fibres (MERRF); Neurogenic muscle weakness, ataxia and retinitis pigmentosa (NARP); Chronic progressive external opthalmoplegia (CPEO); and Mitochondrial neurogastro-intestinal encephalopathy (MNGIE) syndrome.

Another group of diseases that can be treated by therapeutic interventions involving HSF1 pathway are lysosomal storage diseases (LSD). LSD are disorders that are driven by genetic mutations that result in dysfunctional lysosomal proteins—primarily lysosomal hydrolases and membrane proteins. Lysosomes are vital for the maintenance of cellular homeostasis by recycling cell constituents. Also, the severity of lysosomal storage diseases are indicative of the vital nature of lysosomal function. Clinical phenotypes associated with LSDs include severe neurodegeneration, systemic disease and early death driven by protein misfolding and aggregation, impaired lysosomal trafficking and autophagy, oxidative stress, endoplasmic reticulum stress response, impaired calcium homeostasis, and loss of lysosomal stability (Platt. Nat Rev Drug Discov. 2018, 17, 133-150; Ingemann, Kirkegaard. J Lipid Res. 2014, 55, 2198-2210).

Activation of HSF1 inhibits protein misfolding and aggregation resulting from genetic mutations, and the upregulation of heat shock chaperones have been shown to improve enzymatic function of misfolded proteins by refolding misfolded proteins. Other benefits from the activation of HSF1 include enhancing cellular survival by inhibiting lysosomal membrane permeabilization, and increasing lysosomal catabolism (Ingemann, Kirkegaard. J Lipid Res. 2014, 55, 2198-2210).

A group of diseases that can be treated by therapeutic interventions involving HSF1 pathway are LSD including: GM1-gangliosidosis, GM2-gangliosidosis, Alpha-mannosidosis, Beta-mannosidosis, Aspartylglucosaminuria, Lysosomal acid lipase deficiency, Wolman disease, Cystinosis, Chanarin-Dorfman syndrome, Danon disease, Fabry disease (type I and II), Faber disease, Fucosidosis, Galactosialidosis, Gaucher disease (type I, II, III, IIIC, Saposin C deficiency), Krabbe disease, Metachromatic Leukodystrophy, Hurler syndrome, Hurler-Scheie syndrome, Scheie syndrome, Hunter syndrome, Sanfilippo syndrome Type A, Sanfilippo syndrome Type B, Sanfilippo syndrome Type C, Sanfilippo syndrome Type D, Morquio syndrome, type A, Morquio syndrome, type B, Hyaluronidase deficiency, Maroteaux-Lamy syndrome, Sly syndrome, Sialidosis, Leroy disease, Pseudo-Hurler polydystrophy, Mucolipidosis IIIC, Mucolipidosis type IV, Multiple sulfatase deficiency, Niemann-Pick disease (Type A, B, C1, C2 and D), CLN6 disease—Atypical Late Infantile, Late-Onset variant, Early Juvenile; Batten-Spielmeyer-Vogt/Juvenile NCL/CLN3 disease, Finnish Variant Late Infantile CLN5, Jansky-Bielschowsky disease/Late infantile CLN2/TPP1 disease, Kufs/Adult-onset NCL/CLN4 disease, Northern Epilepsy/variant late infantile CLN8, Santavuori-Haltia/Infantile CLN1/PPT disease, Pompe disease (glycogen storage disease type II), Pycnodysostosis, Sandhoff disease (infantile, juvenile, and adult onset), Schindler disease (Type I, III), Schindler disease Type II/Kanzaki disease, Salla disease, Infantile free sialic acid storage disease, Spinal muscular atrophy with progressive myoclonic epilepsy (SMAPME), Tay-Sachs disease, Christianson syndrome, Lowe oculocerebrorenal syndrome, Charcot-Marie-Tooth, Yunis-Varon syndrome, Bilateral temporooccipital polymicrogyria (BTOP), and X-linked hypercalciuric nephrolithiasis.

Another group of diseases that can be treated by therapeutic interventions involving HSF1 pathway are tauopathies. Tauopathies are neurodegenerative disorders that are driven by intracellular tau protein misfolding and aggregation caused by genetic mutations in the MAPT gene and/or post translational modification of tau protein. Tau protein aggregates have been demonstrated to have a correlation with cognitive decline in multiple neurodegenerative diseases. And hyperphosphorylated soluble Tau and insoluble Tau proteins have both been shown to be neurotoxic. In fact, soluble hyperphosphorylated Tau is taken up in neurons, and serves as template for cytoplasmic Tau misfolding. Further studies have also indicated that Tau protein aggregation is driven by aberrant liquid-liquid phase separation/stress granules which persist and enhances aggregation.

Activation of HSF1 has been shown to upregulate molecular chaperones that inhibit tau protein misfolding and aggregation, refold misfolded tau proteins, disaggregate tau oligomers and aggregates, (Patterson et al. Biochemistry. 2011, 50, 10300-10310; Baughman et al. J. Biol. Chem. 2018, 293, 2687-2700), and degrade terminally aggregated tau proteins via the ubiquitin proteasome system and autophagy including chaperone mediated autophagy. (Boland et al. Nat Rev Drug Discov. 2018, 17, 660-688). HSF1 activation also improves synaptic plasticity, neuronal survival and neurotransmitter release at the synapse. (Gomez-Pastor et al. Nat Rev Mol Cell Biol. 2018, 19, 4-19). The upregulation of molecular chaperones has been demonstrated to inhibit protein misfolding and aggregation, refold misfolded proteins, disaggregate aggregated proteins, and degrade terminally misfolded and aggregated proteins via autophagy. These proteins include α-Synuclein, TDP-43, FUS, ATXN2, Amyloid β, polyglutamine (polyQ) expansion proteins, polytrinucleotide repeat expansions, polyhexanucleotide repeat expansions, misfolded SOD1 and several other misfolded proteins resulting from genetic mutations.

Diseases that are primarily driven by MAPT genetic mutations and/or post translational modifications of tau include: Progressive supranuclear palsy, Corticobasal degeneration, Pick's disease, Frontotemporal lobar degeneration-tau, Argyrophilic grain disease, Subacute sclerosing panencephalitis, Christianson syndrome, Post-encephalitic parkinsonism, Guadeloupean parkinsonism, Spinocerebellar ataxia type 11, Chronic traumatic encephalopathy, Aging-related tau astrogliopathy (ARTAG), Globular glial tauopathy, and Primary age-related tauopathy (PART).

Diseases/disorders where tau plays a significant role in the diseases but are primarily driven by β-Amyloid include: Alzheimer's disease, Cerebral amyloid angiopathy, Vascular dementia, Down's syndrome.

Diseases/disorders where tau plays a significant role in the diseases but are primarily driven by α-Synuclein include: Parkinson's disease, Dementia with Lewy bodies, Parkinson's disease dementia, Neurodegeneration with brain iron accumulation, Diffuse neurofibrillary tangles with calcification, Multiple systems atrophy, and Alzheimer's disease.

Diseases/disorders where tau plays a significant role in the diseases but are primarily driven by Prion include: Creutzfeldt-Jakob disease, Fatal familial insomnia, Gerstmann-Straussler-Scheinker syndrome, and Kuru.

Diseases/disorders where tau plays a significant role in the diseases but are primarily driven by other factors include: Huntington's disease, Familial British dementia, Familial Danish dementia, Parkinsonism-dementia of Guam, Frontotemporal lobar degeneration-C9ORF72, Myotonic dystrophy, Niemann-Pick disease type C, Neuronal ceroid lipofuscinosis, and Inclusion body myositis.

Other hereditary diseases can also be treated by therapeutic interventions involving the HSF1 pathway. Hereditary diseases are diseases/disorders that are driven by genetic mutations that result in dysfunctional proteins, and cause loss-of-function of the translated protein. These diseases tend to result in heterogenous clinical phenotypes. Activation of the heat shock response primarily through HSF1 activation produces multiple molecular chaperones that attenuate protein misfolding, and refold the dysfunctional proteins to restore some, if not all, protein/enzyme function. Thus, slowing and/or inhibiting disease progression.

Diseases that are typically hereditary diseases include: Alexander disease, Aortic medial amyloidosis, ApoAI amyloidosis, ApoAII amyloidosis, ApoAIV amyloidosis, Autosomal Dominant Hyper-IgE syndrome, Bloom's syndrome, Brown-Vialetto-Van Laere syndrome, Cockayne's syndrome, Cushing's disease, Cystic fibrosis, Dentatorubropallidoluysian Atrophy (DRPLA), Duchenne's paralysis, Eales Disease, Familial amyloidosis of the Finnish type, Familial amyloidotic neuropathy, Familial dementia, Fragile X syndrome, Fragile X-associated Tremor/Ataxia Syndrome (FXTAS), Friedreich's ataxia, Glycogen Storage Disease type IV (Andersen Disease), Hereditary lattice corneal dystrophy, Hereditary Leber Optic Atrophy, hereditary spastic paraplegia (HSP), Hutchinson-Guilford disease, Kugelberg-Welander syndrome, Lou Gehrig's disease, light chain or heavy chain amyloidosis, Mallory bodies, Paget's disease of the bone (PDB), Pelizaeus-Merzbacher disease, primary lateral sclerosis (PLS), Senger's syndrome, Sickle cell disease, Spinal and bulbar muscular atrophy (SBMA) (also known as Kennedy's disease), Variant Creutzfeldt-Jakob Disease, Werdnig-Hoffmann disease, Werner syndrome.

Other Protein misfolding and age-related diseases can also be treated by therapeutic interventions involving HSF1 pathway. Heat shock response is a cytoprotective response mechanism in cells, including neurons, that are under cell stress. In several degenerative diseases, including neurodegenerative diseases, heat shock response is suboptimal. Furthermore, heat shock response in cells in response to stress diminishes with age, and has been shown to be the cause of several degenerative diseases (Klaips et al. J Cell Biol. 2018, 217, 51-63; Chiti, Dobson. Annu. Rev. Biochem. 2017, 86, 27-68; Labbadia, Morimoto. Annu. Rev. Biochem. 2015, 84, 435-464; Morimoto. Cold Spring Harb Symp Quant Biol. 2011, 76, 91-99).

Activation of the heat shock response primarily through HSF1 activation produces multiple molecular chaperones that inhibit protein misfolding and aggregation, refold misfolded proteins and disaggregate aggregated proteins. HSF1 activation also reduces oxidative stress, improves mitochondrial function and initiates mitochondrial biogenesis, improves synaptic plasticity, and neuronal survival. (Gomez-Pastor et al. Nat Rev Mol Cell Biol. 2018, 19, 4-19).

Protein misfolding and age-related diseases include: Amyotrophic Lateral Sclerosis, ataxia and retinitis pigmentosa, ataxia neuropathy spectrum, ataxia telangiectasia, atherosclerosis, atrial fibrillation, autism spectrum disorder, benign focal amyotrophy, cardiac atrial amyloidosis, cardiovascular diseases (including coronary artery disease, myocardial infarction, stroke, restenosis and arteriosclerosis), cataracts, cerebral hemorrhage, cerebrovascular accident, corneal lactoferrin amyloidosis, Critical Illness Myopathy (CIM), Crohn's Disease, cutaneous lichen amyloidosis, demyelinating disorders, Dentatorubropallidoluysian Atrophy (DRPLA), depressive disorder, diabetes type II, dialysis amyloidosis, endotoxin shock, fibrinogen amyloidosis, glaucoma, ischemia, ischemic conditions (including ischemia/reperfusion injury, myocardial ischemia, stable angina, unstable angina, stroke, ischemic heart disease and cerebral ischemia), lactic acidosis and stroke-like episodes (MELAS) syndrome, lysozyme amyloidosis, macular degeneration, medullary thyroid carcinoma, meningitis and encephalitis, multiple sclerosis, necrotizing enterocolitis, neurofibromatosis, odontogenic (Pinborg) tumor amyloid, pituitary prolactinoma, post-traumatic stress disorder, presenile dementia, prion diseases (also known as Transmissible Spongiform Encephalopathies or TSEs, including Creutzfeldt-Jakob Disease (CJD), progressive bulbar palsy (PBP), progressive muscular atrophy (PMA), pseudobulbar palsy, pulmonary alveolar proteinosis, retinal ganglion cell degeneration in glaucoma, retinal ischemia, retinal vasculitis, retinitis pigmentosa with rhodopsin mutations, schizophrenia, seminal vesical amyloid, senile cataract, senile systemic amyloidosis, Serpinopathies, subarachnoid hemorrhage, temporal lobe epilepsy, transient ischemic attack, ulcerative colitis, and Valosin-Containing Protein (VCP)-related disorders.

The transcription nuclear erythroid 2—related factor 2 (NRF2, HGNC:7782, https://www.ncbi.nlm.nih.gov/gene/4780) regulates the expression of genes involved in cellular protection against damage by oxidants, electrophiles, and inflammatory agents, and in the maintenance of mitochondrial function, cellular redox and protein homeostasis. NRF2 protein comprises seven functional domains, named NRF2-ECH homology (Neh) 1-7 domains. NRF2 binds one of its major negative regulators, Kelch-like ECH-associated protein 1 (Keap1) through its Neh2 domain. In addition, Neh1 is responsible for the formation of a heterodimer with small musculoaponeurotic fibrosarcoma (sMaf) proteins, and mediates binding to antioxidant/electrophile response element (ARE/EpRE) sequences in the promoter regions of Nrf2 target genes. The C-terminus Neh3 is another transactivation domain that recruits chromo-ATPase/helicase DNA-binding protein 6 (CHD6). Neh4 and Neh5 are transactivation domains that recruit cAMP response element-binding protein (CREB)-binding protein (CBP) and/or receptor-associated coactivator 3 (RAC3). The Neh6 domain mediates interaction with a third negative regulator, β-transducin repeat-containing protein (β-TrCP). Moreover, the Neh7 domain mediates binding to retinoid X receptor alpha (RXRα), another negative regulator of NRF2.

NRF2 levels are regulated primarily by ubiquitination and proteasomal degradation. After binding to the Neh2 domain, Keap1 mediates the Cullin3 (Cul3)/Rbx1-dependent ubiquitination of NRF2. Additionally, the Neh6 domain contains a phosphodegron for β-TrCP/Cullin1-mediated ubiquitination. Synoviolin (Hrd1) and WDR23-DDB1-Cul4 are two other ubiquitin ligases that have been shown to participate in the proteasomal degradation of NRF2.

At homeostatic conditions, NRF2 is a short-lived protein. Under stress conditions, NRF2 is stabilized and translocates to the nucleus, where it binds to the ARE/EpRE sequences in the promoter of its target genes, and activates their transcription. NRF2 targets include genes that encode detoxification, antioxidant, and anti-inflammatory proteins as well as proteins involved in the regulation of autophagy and clearance of damaged proteins, such as proteasomal subunits. Activation of NRF2 leads to the upregulation of proteins involved in the synthesis of glutathione, the main intracellular small molecule antioxidant, and NADPH, which provides reducing equivalents for the regeneration of reduced glutathione (GSH) from its oxidized form, GSSG. NRF2 also participates in the maintenance of mitochondrial function and quality control, through activation of mitophagy. NRF2 inhibits the transcription of genes encoding pro-inflammatory cytokines and suppresses pro-inflammatory responses following exposure to ultraviolet radiation or lipopolysaccharide. Such comprehensive cytoprotective functions suggest potential benefits of therapeutic targeting of NRF2 to counteract neurodegeneration.

NRF2 activators have pleiotropic effects on multiple neurodegenerative disease pathways and show great promise for neuroprotection in these disorders. As a NRF2 activator, (6aR)-6-methyl-5,6,6a,7-tetrahydro-4H-dibenzo[de,g]quinoline-10,11-diol has beneficial effects on key drivers of neurodegeneration, including redox imbalance, inflammation, mitochondrial dysfunction and altered proteostasis/autophagy in the pathogenesis.

The pharmacological activation of NRF2 and the NRF2 pathway is another promising avenue for therapeutic intervention in diseases involving the NRF2 pathway. NRF2 activators represents a novel therapeutic strategy that could slow, halt, or reverse the underlying disease process in diseases involving the NRF2 pathway.

The combined pharmacological activation of HSF1 and NRF2, and the HSF1 and NRF2 pathways, is another promising avenue for therapeutic intervention in diseases involving both the HSF1 and NRF2 pathways. Combined HSF1/NRF2 activators represents a novel therapeutic strategy that could slow, halt, or reverse the underlying disease process in diseases involving the HSF1 and NRF2 pathways.

Accordingly, there is a need for compositions with have utility in the activation of HSF1 and/or NRF2.

SUMMARY OF THE INVENTION

6-Methyl-5,6,6a,7-tetrahydro-4H-dibenzo[de,g]quinoline-10,11-diol is a type of aporphine having activity on dopamine receptors. It may also have effects on serotonergic and adrenergic receptors. 6-Methyl-5,6,6a,7-tetrahydro-4H-dibenzo[de,g]quinoline-10,11-diol does not actually contain morphine or its skeleton, nor does it bind to opioid receptors. The apo-prefix relates to it being a morphine derivative. 6-Methyl-5,6,6a,7-tetrahydro-4H-dibenzo[de,g]quinoline-10,11-diol has two enantiomers, i.e., (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol. (6aR)-6-methyl-5,6,6a,7-tetrahydro-4H-dibenzo[de,g]quinoline-10,11-diol is a central nervous system penetrant catechol amine compound that is a dual activator of HSF1 and NRF2 transcription factors.

(6aR)-6-methyl-5,6,6a,7-tetrahydro-4H-dibenzo[de,g]quinoline-10,11-diol is a strong dopamine agonist and currently approved for the treatment of Parkinson's disease. (6aR)-6-methyl-5,6,6a,7-tetrahydro-4H-dibenzo[de,g]quinoline-10,11-diol, also known as R-(−)-10,11-dihydroxyaporphine, is depicted by the following chemical structure:

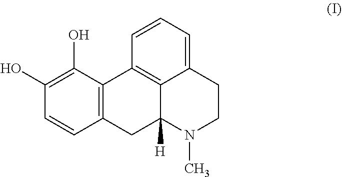 [SMILES: CN1CCc2cccc3c2[C@H]1Cc1ccc(O)c(O)c1-3]

(6aS)-6-methyl-5,6,6a,7-tetrahydro-4H-dibenzo[de,g]quinoline-10,11-diol, the enantiomer of (6aR)-6-methyl-5,6,6a,7-tetrahydro-4H-dibenzo[de,g]quinoline-10,11-diol, is a weak dopamine antagonist and does not exhibit the side effects associated with dopamine agonism after administration. (6aS)-6-methyl-5,6,6a, 7-tetrahydro-4H-dibenzo[de,g]quinoline-10,11-diol, also known as S-(+)-10,11-dihydroxyaporphine, is depicted by the following chemical structure:

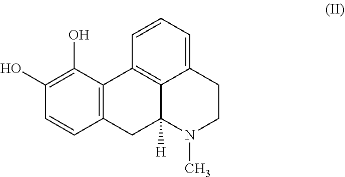 [SMILES: CN1CCc2cccc3c2[C@@H]1Cc1ccc(O)c(O)c1-3]

The present invention is predicted on the surprising finding, demonstrated through screens and tests, that 6-methyl-5,6,6a,7-tetrahydro-4H-dibenzo[de,g]quinoline-10,11-diol is a potent HSF1 activator and can significantly impact protein misfolding, accumulation of misfolded proteins, and protein aggregation.

Accordingly, 6-methyl-5,6,6a,7-tetrahydro-4H-dibenzo[de,g]quinoline-10,11-diol may be used in methods to activate HSF1, to increase the level of transcription of genes positively regulated by HSF1, i.e., activate the HSF1 pathway, to increase the cellular level of protein chaperones and/or co-chaperones, to reduce the frequency of protein misfolding, to reduce the accumulation of misfolded proteins, and to reduce protein aggregation in a cell. 6-Methyl-5,6,6a,7-tetrahydro-4H-dibenzo[de,g]quinoline-10,11-diol may further be used in methods for treating diseases mediated by protein misfolding, accumulation of misfolded proteins, protein aggregation, or by reduced HSF1 activity. HSF1 activation by 6-methyl-5,6,6a,7-tetrahydro-4H-dibenzo[de,g]quinoline-10,11-diol is determined by the upregulation of HSF1 target genes.

Specifically, (6aS)-6-methyl-5,6,6a,7-tetrahydro-4H-dibenzo[de,g]quinoline-10,11-diol and (6aR)-6-methyl-5,6,6a,7-tetrahydro-4H-dibenzo[de,g]quinoline-10,11-diol are converted to the ortho-quinone moiety under oxidative stress a pathomechanism of neurodegenerative diseases. (6aS)-6-methyl-5,6,6a,7-tetrahydro-4H-dibenzo[de,g]quinoline-10,11-diol is classified as a pro-electrophilic and pathologically activated drug and is an electrophile 3 compound. (Satoh et al. Free Radic Biol Med. 2013, 65, 645-657; Satoh et al. J Neurochem. 2011, 119, 569-578; Satoh et al. ASN Neuro. 2015, 1-13).

The ortho-quinone moiety of (6aS)-6-methyl-5,6,6a,7-tetrahydro-4H-dibenzo[de,g]quinoline-10,11-diol serves as a Michael acceptor which undergoes Michael additions with specific cysteine residues of regulators of transcription factors. On formation of the Hsp90-(6aS)-6-methyl-5,6,6a,7-tetrahydro-4H-dibenzo[de,g]quinoline-10,11-diol ortho-quinone adduct under cellular stress, Hsf1, a monomer, is released from the Hsp90, Hsp70, Hsp40, Hsf1, and TRiC complex, homotrimerizes, and translocates to the nucleus where in binds to heat shock elements (HSE) to activate the transcription and translation of downstream genes that enhance neuronal survival and function. (Gomez-Pastor et al. Nat Rev Mol Cell Biol. 2018, 19, 4-19; Naidu, Dinkova-Kostova. FEB S J. 2017, 284, 1606-1627). The genes activated, which has been estimated to be between 50-200 genes, encode protein chaperones (including heat shock protein 70 (Hsp70), Synapsin, postsynaptic density protein 95 (PSD95), brain-derived neurotrophic factor (BDNF), and peroxisome proliferator-activated receptor-γ coactivator 1α (PGC-1α).

The protein chaperones, including Hsp70 and Hsp40, and heat shock cognate 70/heat shock protein A8 (Hsc70/HSPA8), are responsible for inhibiting protein misfolding and aggregation, refolding misfolded proteins, disaggregating aggregated proteins, and clearing terminally folded and/or aggregated proteins via the ubiquitin proteasome system (UPS) and autophagy, including chaperone-mediated autophagy (CMA). These chaperones are also involved in inhibiting the formation of pathological stress granules, disaggregation of pathological stress granules, and clearance of terminally formed aberrant stress granules via autophagy. The disaggregation of pathological stress granules has been demonstrated to restore dysfunctional nucleocytoplasmic transport, which is a pathomechanism in multiple neurodegenerative diseases, via the release of nucleocytoplasmic transport factors trapped in aberrant stress granules.

Hsp70 has been shown to attenuate the formation of pro-inflammatory cytokines via the inhibition of the formation of IkBα phosphorylation which is upstream of the NF-kB signaling pathway during the symptomatic phase of neurodegenerative diseases.

Neurodegenerative diseases that are driven by impaired proteostasis include amyotrophic lateral sclerosis, motor neuron diseases, frontotemporal dementia, all tauopathies including Alzheimer's disease, FTLD-tau, progressive supranuclear palsy, corticobasal degeneration, chronic traumatic encephalopathy (Gomez-Pastor et al. Nat Rev Mol Cell Biol. 2018, 19, 4-19; Li, Gotz, Nat Rev Drug Discov. 2017, 12, 863-883), Parkinson's disease, Dementia with Lewy bodies, pathological polyglutamine expansion diseases including Huntington's disease, Hereditary spastic paraplegia, Spastic ataxia, Marinesco-Sjogren's syndrome, Charcot-Marie Tooth disease type 2L, juvenile parkinsonism, distal hereditary motor neuropathy, dominantly inherited myopathy, Angelman's syndrome, Nakajo-Nishimura syndrome, DCMA syndrome, Desmin related myopathy, spinocerebellar ataxia type 3/Marchado-Joseph disease (SCA3/MJD), and Paget disease. (Labbadia, Morimoto. Annu Rev Biochem. 2015, 84, 435-464). Lysosomal storage disorders including Niemann-Picks Type C, Gaucher, Fabry, Sandhoff, Tay Sachs, Wolman, Pompe, Mucolipidosis type II, Mucolipidosis type IV, Multiple sulfatase deficiency, Galactosialidosis, Neuronal ceroid lipofuscinosis, Mucopolysaccharidosis type I, Mucopolysaccharidosis type II, Mucopolysaccharidosis type III, Mucopolysaccharidosis type IV, and Metachromatic leukodystrophy (Platt. Nat Rev Drug Discov. 2018, 17, 133-150; Ingemann, Kirkegaard. J Lipid Res. 2014, 55, 2198-2210), and Sporadic inclusion body myositis (Ahmed et al. Sci Transl Med. 2016, 8, 331).

In one aspect, the present invention provides a method of activating HSF1 in a cell, comprising a step of contacting the cell with an effective amount of 6-methyl-5,6,6a,7-tetrahydro-4H-dibenzo[de,g]quinoline-10,11-diol. As used herein, the term “effective amount” means an amount that will result in the desired effect or result, e.g., an amount that will result in activating HSF1.

In a related aspect, the present invention provides a method of increasing transcription of a gene that is transactivated by HSF1 in a cell, comprising the step of contacting said cell with an effective amount of 6-methyl-5,6,6a,7-tetrahydro-4H-dibenzo[de,g]quinoline-10,11-diol.

In another aspect, the present invention provides a method of increasing the cellular level of a protein chaperones and/or co-chaperones in a cell, comprising the step of contacting said cell with an effective amount of 6-methyl-5,6,6a,7-tetrahydro-4H-dibenzo[de,g]quinoline-10,11-diol.

In another aspect, the present invention provides a method of reducing the frequency of protein misfolding or accumulation of misfolded proteins including TDP-43, SOD1, hyperphosphorylated Tau, dipeptide repeat proteins from hexanucleotide repeat expansion associated with C9orf72, β-amyloid, α-synuclein, phosphorylated α-synuclein, polyglutamine repeat expansion, FUS, ATXN2, heterogeneous nuclear ribonucleoproteins (hnRNPs), and prion proteins in a cell, comprising the step of contacting said cell with an effective amount of 6-methyl-5,6,6a,7-tetrahydro-4H-dibenzo[de,g]quinoline-10,11-diol.

In another aspect, the invention provides a method of increasing cell lifespan, comprising the step of contacting said cell with an effective amount of 6-methyl-5,6,6a,7-tetrahydro-4H-dibenzo[de,g]quinoline-10,11-diol.

In one embodiment, the cell in one of the above aspects, or other aspect herein, is a cell type or from a tissue selected from any one or more of: adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder and vagina. In a further embodiment, said brain cell is from a brain tissue selected from cerebrum (including cerebral cortex, basal ganglia (often called the striatum), and olfactory bulb), cerebellum (including dentate nucleus, interposed nucleus, fastigial nucleus, and vestibular nuclei), diencephalon (including thalamus, hypothalamus, etc. and the posterior portion of the pituitary gland), and brain-stem (including pons, substantia nigra, medulla oblongata). In a further embodiment, said brain cell is selected from a neuron or glia cell (e.g., an astrocyte, oligodendrocyte, or microglia). In a further embodiment, said neuron is a sensory neuron, motor neuron, interneuron, or brain neuron.

In one embodiment, the cell is an animal cell, e.g., mammalian cell. In a further embodiment, said cell in a human cell or non-human cell. In a further embodiment, said cell is in vitro, in vivo, or ex vivo.

In another embodiment, the cell is a diseased cell. In another embodiment, the cell is diseased cell from a patient suffering from a disease or disorder below.

In another aspect, the invention provides a method of treating an animal having a disease or disorder that would benefit from increased HSF1 activation, or for preventing or reducing the risk of acquiring a disease or disorder in an animal, the method comprising the step of administering a therapeutically effective amount of a pharmaceutical composition comprising 6-methyl-5,6,6a,7-tetrahydro-4H-dibenzo[de,g]quinoline-10,11-diol to said animal. In one embodiment, said animal is a mammal. In another embodiment, said mammal is a human or a non-human mammal. In a further embodiment, said mammal is a human. In another embodiment, said disease or disorder is caused by protein misfolding, accumulation of misfolded proteins, or protein aggregation. In another embodiment, said disease is selected from any one or more of: aging-related tau astrogliopathy (ARTA), Alexander Disease, Alpers-Huttenlocher syndrome, Alzheimer's disease, Amyotrophic Lateral Sclerosis (ALS), Ataxia neuropathy spectrum, ataxia and retinitis pigmentosa (NARP), Critical Illness Myopathy (CIM), Primary Age-Related Tauopathy (PART), aortic medial amyloidosis, ApoAI amyloidosis, ApoAII amyloidosis, ApoAIV amyloidosis, argyrophillic grain disease, ataxia telangiectasia, atrial fibrillation, Autosomal Dominant Hyper-IgE Syndrome, cardiac atrial amyloidosis, Bloom's syndrome, cardiovascular diseases (including coronary artery disease, myocardial infarction, stroke, restenosis and arteriosclerosis), cataracts, cerebral amyloid angiopathy, Christianson syndrome, chronic traumatic encephalopathy, Chronic progressive external opthalmoplegia (CPEO), Cockayne's syndrome, congenital lactic acidosis (CLA), corneal lactoferrin amyloidosis, corticobasal degeneration, Crohn's Disease, Cushing's disease, cutaneous lichen amyloidosis, cystic fibrosis, Dentatorubropallidoluysian Atrophy (DRPLA), dialysis amyloidosis, diffuse neurofibrillary tangles with calcification, Down syndrome, endotoxin shock, familial amyloidosis of the Finnish type, familial amyloidotic neuropathy, Familial British Dementia (FBD), Familial Danish Dementia (FDD), familial dementia, fibrinogen amyloidosis, fragile X syndrome, Fragile X-associated Tremor/Ataxia Syndrome (FXTAS), Friedreich's ataxia, frontotemporal degeneration, glaucoma, Glycogen Storage Disease type IV (Andersen Disease), Guadeloupean Parkinsonism, hereditary lattice corneal dystrophy, Huntington's disease, inclusion body myositis/myopathy, inflammation, inflammatory bowel disease, ischemic conditions (including ischemia/reperfusion injury, myocardial ischemia, stable angina, unstable angina, stroke, ischemic heart disease and cerebral ischemia), light chain or heavy chain amyloidosis, lysosomal storage diseases (including aspartylglucosaminuria), Fabry's disease, Batten disease, Cystinosis, Farber, Fucosidosis, Galactasidosialidosis, Gaucher's disease (including Types 1, 2 and 3), Gml gangliosidosis, Hunter's disease, Hurler-Scheie's disease, Krabbe's disease, a-Mannosidosis, Kearns-Sayre syndrome (KSS), lactic acidosis and stroke-like episodes (MELAS) syndrome, Leber hereditary optic neuropathy (LHON), B-Mannosidosis, Maroteaux-Lamy's disease, MEGDEL syndrome (also known as 3-methylglutaconic aciduria with deafness, encephalopathy and Leigh-like syndrome), Metachromatic Leukodystrophy, Mitochondrial neurogastro-intestinal encephalopathy (MNGIE) syndrome, Morquio A syndrome, Morquio B syndrome, Mucolipidosis II, Mucolipidosis III, Myoclonic epilepsy myopathy sensory ataxia, Mitochondrial myopathy, Myoclonic epilepsy with ragged red fibres (MERRF), Niemann-Pick Disease (including Types A, B and C), Neurogenic muscle weakness, Pearson syndrome, Pompe's disease, Sandhoff disease, Sanfilippo syndrome (including Types A, B, C and D), Schindler disease, Schindler-Kanzaki disease, Sengers syndrome, Sialidosis, Sly syndrome, Tay-Sach's disease, Wolman disease, lysozyme amyloidosis, Mallory bodies, medullary thyroid carcinoma, mitochondrial myopathies, multiple sclerosis, multiple system atrophy, myotonic dystrophy, myotonic dystrophy, neurodegeneration with brain iron accumulation, neurofibromatosis, neuronal ceroid lipofuscinosis, odontogenic (Pinborg) tumor amyloid, Parkinsonism-Dementia of Guam, Parkinson's disease, peptic ulcers, Pick's disease, pituitary prolactinoma, post-encephalitic Parkinsonism, prion diseases (also known as Transmissible Spongiform Encephalopathies or TSEs, including Creutzfeldt-Jakob Disease (CJD), Variant Creutzfeldt-Jakob Disease, Gerstmann-Straussler-Scheinker Syndrome, Fatal Familial Insomnia, and Kuru), progressive supranuclear palsy, pulmonary alveolar proteinosis, retinal ganglion cell degeneration in glaucoma, retinitis pigmentosa with rhodopsin mutations, seminal vesical amyloid, senile systemic amyloidoses, Serpinopathies, sickle cell disease, spinal and bulbar muscular atrophy (SBMA) (also known as Kennedy's disease), spinocerebellar ataxias (including spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 8, and spinocerebellar ataxia type 17), subacute sclerosing panencephalitis, tauopathies, type II diabetes, vascular dementia, Werner syndrome, atherosclerosis, autism spectrum disorder (ASD), benign focal amyotrophy, Duchenne's paralysis, hereditary spastic paraplegia (HSP), Kugelberg-Welander syndrome, Lou Gehrig's disease, necrotizing enterocolitis, Paget's disease of the bone (PDB), primary lateral sclerosis (PLS), progressive bulbar palsy (PBP), progressive muscular atrophy (PMA), pseudobulbar palsy, spinal muscular atrophy (SMA), ulcerative colitis, Valosin-Containing Protein (VCP)-related disorders, or Werdnig-Hoffmann disease, transient ischemic attack, ischemia, cerebral hemorrhage, senile cataract, retinal ischemia, retinal vasculitis, Brown-Vialetto-Van Laere syndrome, Eales Disease, meningitis and encephalitis, post-traumatic stress disorder, Charcot-Marie-Tooth Disease, macular degeneration, X-Linked Bulbo-Spinal Atrophy, presenile dementia, depressive disorder, temporal lobe epilepsy, Hereditary Leber Optic Atrophy, cerebrovascular accident, subarachnoid hemorrhage, schizophrenia, demyelinating disorders, and Pelizaeus-Merzbacher disease.

In one embodiment, the disease is selected from any one or more of: Lysosomal Storage Diseases (e.g., Niemann-Picks Type C, Gaucher Disease), inclusion body myositis, spinocerebellar ataxias, spinal and bulbar muscular atrophy, or a condition associated therewith.

In another embodiment, said disease is a neurological disease.

In another embodiment, the disease is selected from any one or more of: amyotrophic lateral sclerosis, frontotemporal dementia, Huntington's disease, Alzheimer's disease, Parkinson's disease, dementia with Lewy bodies, Parkinson's disease dementia, neurodegeneration with brain iron accumulation, diffuse neurofibrillary tangles with calcification, multiple system atrophy, cerebral amyloid angiopathy, vascular dementia, Down's syndrome, Creutzfeldt-Jakob disease, fatal familial insomnia, Gerstmann-Straussler-Scheinker syndrome, kuru, familial British dementia, familial Danish dementia, Parkinsonism-Dementia of Guam, myotonic dystrophy, neuronal ceroid lipofuscinosis, or a condition associated therewith.

In another embodiment, the disease is selected from any one or more of: frontotemporal dementia, neurodegeneration with brain iron accumulation, diffuse neurofibrillary tangles with calcification, multiple system atrophy, cerebral amyloid angiopathy, vascular dementia, Down's syndrome, Creutzfeldt-Jakob disease, fatal familial insomnia, Gerstmann-Straussler-Scheinker syndrome, kuru, familial British dementia, familial Danish dementia, myotonic dystrophy, neuronal ceroid lipofuscinosis, or a condition associated therewith.

In another embodiment, the disease is selected from Friedreich's ataxia, multiple sclerosis, mitochondrial myopathies, progressive supranuclear palsy, corticobasal degeneration, chronic traumatic encephalopathy, argyrophillic grain disease, subacute sclerosing panencephalitis, Christianson syndrome, post-encephalitic Parkinsonism, Guadeloupean Parkinsonism, aging-related tau astrogliopathy (ARTA), and primary age-related tauopathy (PART), Pick's disease, or a condition associated therewith.

In another aspect, the invention provides a method of increasing lifespan or treating a disease or disorder resulting in accelerated aging or other abnormal aging process in an animal, the method comprising the step of administering a therapeutically effective amount of a pharmaceutical composition comprising 6-methyl-5,6,6a,7-tetrahydro-4H-dibenzo[de,g]quinoline-10,11-diol to said animal. In one embodiment, said animal is a mammal. In another embodiment, said mammal is a human or a non-human mammal. In one embodiment, said disease or disorder is selected from Werner syndrome, Hutchinson-Guilford disease, Bloom's syndrome, Cockayne's syndrome, ataxia telangiectasia, and Down syndrome.

In a related aspect, the invention provides a method of treating premature aging due to chemical or radiation exposure. In one embodiment, the premature aging is due to exposure to chemotherapy, radiation therapy, or UV radiation. In a further embodiment, the UV radiation is artificial, e.g., tanning bed, or solar UV radiation, i.e., sun exposure.

In another aspect, the invention provides an in vitro method of screening a candidate therapeutic agent(s) for its ability to activate the HSF1 pathway, the method comprising:
(1) exposing induced astrocytes derived from fibroblast stem cells to a candidate therapeutic;
(2) comparing amounts of misfolded SOD1 between said induced astrocytes exposed to said candidate therapeutics and control cells, e.g., induced astrocytes that are not exposed to said candidate therapeutic (unexposed induced astrocytes).

6-methyl-5,6,6a,7-tetrahydro-4H-dibenzo[de,g]quinoline-10,11-diol may also be used in methods to activate NRF2 and/or to reduce oxidative stress in a cell. (6aS)-6-methyl-5,6,6a,7-tetrahydro-4H-dibenzo[de,g]quinoline-10,11-diol may further be used in methods for treating diseases or disorders mediated by increased oxidative stress or by reduced NRF2 activity. 6-methyl-5,6,6a,7-tetrahydro-4H-dibenzo[de,g]quinoline-10,11-diol may further be used in methods for reducing inflammation or treating diseases or disorders mediated by inflammation.

Activation of the NRF2 transcription factor, via the binding of 6-methyl-5,6,6a,7-tetrahydro-4H-dibenzo[de,g]quinoline-10,11-diol with cysteine residue on Keap1 results in the release of NRF2 which is translocated to the nucleus, binds to antioxidant response elements (ARE), and drives the transcription and translation of over 250 downstream genes which encode for proteins that reduce oxidative stress, provides anti-inflammatory response, improves mitochondrial function and biogenesis, and autophagic removal of terminally misfolded and aggregated neurotoxic proteins. (Dinkova-Kostova et al. FEBS J. 2018, doi: 10.1111/febs.14379).

To overcome some of the technical barriers of measuring NRF2 directly (including a lack of sensitive antibodies for the detection of the low-abundance NRF2 protein and relative stability of NRF2 mRNA during activation of the pathway), researchers have developed novel strategies for monitoring the activity of the NRF2 pathway. Such strategies include (a) the use of stable reporter cell lines in which the expression of luciferase is controlled by one or more ARE sequences, (b) automated, high-content imaging of cell lines expressing fluorescent-tagged Nrf2 or target gene products, and (c) transcriptomic analysis of dynamic changes in gene signatures that have been shown (for example, in ChIP data) to be representative of the battery of Nrf2-regulated genes (Mutter et al., Biochem Soc Trans. 2015, 43, 657-662).

Activation of the NRF2 transcription factor has been shown to modulate the pathomechanisms and provide neuroprotection in neurodegenerative diseases, including Amyotrophic lateral sclerosis, Frontotempral lobar degeneration/Frontotemporal dementia, Alzheimer's disease, Parkinson's disease, Huntington's disease, Friedreich's Ataxia, and Multiple Sclerosis (Dinkova-Kostova et al. FEBS J. 2018, doi: 10.1111/febs.14379; Cuadrado et al. Pharmacol Rev. 2018, 70, 348-383; Dinkova-Kostova, Kazantsev. Neurodegener. Dis. Manag. 2017, 7, 97-100; Johnson, Johnson. Free Radic Biol Med. 2015, 88, 253-267).

In one aspect, the present invention provides for a method of activating NRF2 in a cell, comprising a step of contacting the cell with an effective amount of 6-methyl-5,6,6a,7-tetrahydro-4H-dibenzo[de,g]quinoline-10,11-diol.

In a related aspect, the present invention provides for a method of increasing transcription of a gene that is transactivated by NRF2 in a cell, comprising the step of contacting said cell with an effective amount of 6-methyl-5,6,6a,7-tetrahydro-4H-dibenzo[de,g]quinoline-10,11-diol.

In one embodiment, the cell in one of the above aspects, or other aspect herein, is a cell type or from a tissue selected from any one or more of: adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder and vagina. In a further embodiment, said brain cell is from a brain tissue selected from cerebrum (including cerebral cortex, basal ganglia (often called the striatum), and olfactory bulb), cerebellum (including dentate nucleus, interposed nucleus, fastigial nucleus, and vestibular nuclei), diencephalon (including thalamus, hypothalamus, etc. and the posterior portion of the pituitary gland), and brain-stem (including pons, substantia nigra, medulla oblongata). In a further embodiment, said brain cell is selected from a neuron or glia cell (e.g., an astrocyte, oligodendrocyte, or microglia). In a further embodiment, said neuron is a sensory neuron, motor neuron, interneuron, or brain neuron.

In one embodiment, the cell is an animal cell, e.g., mammalian cell. In a further embodiment, said cell in a human cell or non-human cell. In a further embodiment, said cell is in vitro, in vivo, or ex vivo.

In another embodiment, the cell is a diseased cell. In another embodiment, the cell is diseased cell from a patient suffering from a disease or disorder below.

In another aspect, the invention provides for a method of treating an animal having a disease or disorder that would benefit from increased NRF2 activation, or for preventing or reducing the risk of acquiring a disease or disorder in an animal, the method comprising the step of administering a therapeutically effective amount of a pharmaceutical composition comprising 6-methyl-5,6,6a,7-tetrahydro-4H-dibenzo[de,g]quinoline-10,11-diol to said animal. In one embodiment, said animal is a mammal. In another embodiment, said mammal is a human or a non-human mammal. In a further embodiment, said mammal is a human. In another embodiment, said disease or disorder is selected from any one or more of: aging-related tau astrogliopathy (ARTA), ALS, Alzheimer's disease, argyrophillic grain disease, asthma, cerebral amyloid angiopathy, cerebral ischemia Christianson syndrome, chronic obstructive pulmonary disease, chronic traumatic encephalopathy, corticobasal degeneration, Creutzfeldt-Jakob disease, dementia with Lewy bodies, diffuse neurofibrillary tangles with calcification, Down's syndrome, emphysema, familial British dementia, familial Danish dementia, fatal familial insomnia, Friedreich's ataxia, frontotemporal dementia, Gerstmann-Straussler-Scheinker syndrome, Guadeloupean Parkinsonism, Huntington's disease, kuru, mitochondrial myopathies, multiple sclerosis, multiple system atrophy, myotonic dystrophy, neurodegeneration with brain iron accumulation, neuronal ceroid lipofuscinosis, Parkinson's disease dementia, Parkinson's disease, Parkinson's, Parkinsonism-Dementia of Guam, Pick's disease, post-encephalitic Parkinsonism, primary age-related tauopathy (PART), progressive supranuclear palsy, pulmonary fibrosis, sepsis, septic shock, subacute sclerosing panencephalitis, vascular dementia, or a condition associated therewith.

The foregoing and other features and advantages of the invention will become more apparent from the following detailed description, which proceeds with reference to the accompanying drawings. Such description is meant to be illustrative, and not limiting, of the invention. Obvious variants of the disclosed (6aS)-6-methyl-5,6,6a,7-tetrahydro-4H-dibenzo[de,g]quinoline-10,11-diol crystalline complex in the text, including those described by the drawings and examples will be readily apparent to the person of ordinary skill in the art having the present disclosure, and such variants are considered to be a part of the current invention.

### BRIEF DESCRIPTION OF THE DRAWINGS

FIG. 1. HSF1 and NRF2 gene expression result compared to Gapdh after (6aS)-6-methyl-5,6,6a,7-tetrahydro-4H-dibenzo[de,g]quinoline-10,11-diol dosing in the preclinical in vivo model.

FIG. 2. HSF1 and NRF2 gene expression result compared to Actb after (6aS)-6-methyl-5,6,6a,7-tetrahydro-4H-dibenzo[de,g]quinoline-10,11-diol dosing in the preclinical in vivo model.

FIG. 3. HSF1 and NRF2 gene expression result compared to Gapdh of mouse cortex tissue at 6 hours post last dose of (6aS)-6-methyl-5,6,6a,7-tetrahydro-4H-dibenzo[de,g]quinoline-10,11-diol in a 7-day mouse repeated dose study.

FIG. 4. HSF1 and NRF2 gene expression result compared to Gapdh of mouse cortex tissue at 24 hours post last dose of (6aS)-6-methyl-5,6,6a,7-tetrahydro-4H-dibenzo[de,g]quinoline-10,11-diol in a 7-day mouse repeated dose study.

FIG. 5. Hspa1a gene expression of mouse brain tissue post last dose of (6aS)-6-methyl-5,6,6a,7-tetrahydro-4H-dibenzo[de,g]quinoline-10,11-diol in a 4-day mouse repeated dose study. Two-way ANOVA with repeated measures with Dunnett's post-test. * p<0.5.

FIG. 6. Hspa8 gene expression of mouse brain tissue post last dose of (6aS)-6-methyl-5,6,6a,7-tetrahydro-4H-dibenzo[de,g]quinoline-10,11-diol in a 4-day mouse repeated dose study. Two-way ANOVA with repeated measures with Dunnett's post-test. ** p<0.01

FIG. 7. Mouse weights over time. (A) mouse weights for the 3-month cohort plotted as mean +/−SD (n=6). There is no significant difference between the animals in the two (6aS)-6-methyl-5,6,6a,7-tetrahydro-4H-dibenzo[de,g]quinoline-10,11-diol dosing groups and the vehicle dosed animals. (B) Mouse weights for the 6-month cohort plotted as mean +/−SD (n=14). There is a significant decrease in weight of the 2.5 mg/kg dosed animals when compared to the vehicle dosed animals between 121 days of age and 6 months of age. Two-way ANOVA with repeated measures with Dunnett's post-test. * p<0.5, ** p<0.01, *** p<0.001, **** p<0.0001.

FIG. 8. Rotarod performance over time. Rotarod performance over the course of the study plotted as latency to fall mean +/−SD (n=14). There is a significant increase in rotarod performance in the 5 mg/kg (6aS)-6-methyl-5,6,6a,7-tetrahydro-4H-dibenzo[de,g]quinoline-10,11-diol dosing group when compared to the vehicle dosing group. Two-way ANOVA with Dunnett's post-test. * p<0.5.

FIG. 9. Catwalk gait analysis at 3 and 6 months showing (A, B) forelimb and hindlimb base of support (BOS) and (C) percentage of time on diagonal limbs.

FIG. 10. Catwalk gait analysis at 3 and 6 months showing (A) percentage of time on 3 paws and (B) percentage of time on 4 paws.

FIG. 11. Catwalk gait analysis showing change in (A, B) forelimb and hindlimb BOS between 3 months and 6 months of age.

FIG. 12. Catwalk gait analysis showing change in (A) percentage of time spent on diagonal paws and (B) percentage of time spent on 3 paws, between 3 months and 6 months of age. There is a significant difference in the change of percentage of time spent on diagonal paws between the 2.5 mg/kg twice daily dosing group and the vehicle dosing group, in which the vehicle has a decrease in the time spent on diagonal paws between 3 and 6 months and the 2.5 mg/kg twice daily dosing group has a slight increase. There is a significant decrease in the percentage of time spent on 3 paws of the 2.5 mg/kg twice daily dosing group when compared to the vehicle dosing group. One-way ANOVA with Dunnett's post test. * p<0.5. N=8 per group.

FIG. 13. Catwalk gait analysis showing change in percentage of time spent on 4 paws between 3 months and 6 months of age.

FIG. 14. Compound muscle action potential (CMAP) amplitude and repetitive stimulation. CMAP plotted as individual values plus mean +/−SD (n=14). Two-way ANOVA with Sidak's post-test.

FIG. 15. Relative CMAP at 6 months, calculated based on individual CMAP at 3 months.

FIG. 16. Repetitive stimulation at 6 weeks and 3 months of age plotted as a percentage of the first stimulation, mean +/−SD for each stimulation (n=14).

FIG. 17. Repetitive stimulation at 6 months of age plotted as a percentage of the first stimulation, mean +/−SD for each stimulation (n=14).

FIG. 18. qPCR results of 3-month cortex tissues. Mean relative mRNA levels +/−SD from 3-month cohort cortex tissue normalized to Gapdh and vehicle (n=6). Two-way ANOVA with Dunnett's post-tests. * p<0.5, ** p<0.01, **** p<0.0001.

FIG. 19. qPCR results of 6-month cortex tissues. Mean relative mRNA levels +/−SD from 6 month cohort cortex tissue normalized to Gapdh and vehicle (n=7). Two-way ANOVA with Dunnett's post-test. **** p<0.0001.

FIG. 20. Protein quantification data demonstrated that (6aS)-6-methyl-5,6,6a,7-tetrahydro-4H-dibenzo[de,g]quinoline-10,11-diol induced a significant increase in NQO1 after 48 hours treatment at 10 uM in induced astrocytes derived from healthy individuals (CTR, n=3); patients carrying C9orf72 mutations (C9orf72, n=3); sporadic ALS patients (sALS, n=3) and patients carrying SOD1 mutations (SOD1, n=3). Statistical test: One-way ANOVA with multiple comparison test. Statistically, 10 uM (6aS)-6-methyl-5,6,6a,7-tetrahydro-4H-dibenzo[de,g]quinoline-10,11-diol (labelled as Drug) is compared to DMSO treatment. * p<0.1; ** p<0.01.

### DETAILED DESCRIPTION OF THE PREFERRED EMBODIMENTS

Definitions

The term ‘(6aR)-6-methyl-5,6,6a,7-tetrahydro-4H-dibenzo[de,g]quinoline-10,11-diol’ means R-(−)-10,11-dihydroxyaporphine, including prodrug, salts, solvates, hydrates, and co-crystals thereof.

The term ‘(6aS)-6-methyl-5,6,6a,7-tetrahydro-4H-dibenzo[de,g]quinoline-10,11-diol’ means S-(+)-10,11-dihydroxyaporphine, including prodrug, salts, solvates, hydrates, and co-crystals thereof.

The term ‘6-methyl-5,6,6a,7-tetrahydro-4H-dibenzo[de,g]quinoline-10,11-diol’ means (6aR)-6-methyl-5,6,6a,7-tetrahydro-4H-dibenzo[de,g]quinoline-10,11-diol, or (6aS)-6-methyl-5,6,6a,7-tetrahydro-4H-dibenzo[de,g]quinoline-10,11-diol, or racemic form of (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol, including prodrug, salts, solvates, hydrates, and co-crystals thereof.

As used herein, the terms ‘treat’, ‘treating’ or ‘treatment’ means to alleviate, reduce or abrogate one or more symptoms or characteristics of a disease and may be curative, palliative, prophylactic or slow the progression of the disease.

The term “effective amount” means an amount that will result in activation of, as applicable or specified, HSF1 and/or NRF2, and in a desired effect or result. The term ‘therapeutically effective amount’ means an amount of 6-methyl-5,6,6a,7-tetrahydro-4H-dibenzo[de,g]quinoline-10,11-diol, alone or combined with other active ingredients, that will elicit a desired biological or pharmacological response, e.g., effective to prevent, alleviate, or ameliorate symptoms of a disease or disorder; slow, halt or reverse an underlying disease process or progression; partially or fully restore cellular function; or prolong the survival of the subject being treated.

As used herein, the term “HSF1 activation” or “activation of HSF1” means the dissociation of HSF1 from its inhibitory complex (comprising Hsp40, Hsp70, TRiC and Hsp90) in the cytoplasm and accumulation of homotrimeric HSF1 in the nucleus.

As used herein, the term “NRF2 activation” or “activation of NRF2” means the dissociation of NRF2 from its regulator Kelch-like ECH-associated protein 1 (Keap1) in the cytoplasm and accumulation of NRF2 in the nucleus.

The term ‘patient’ or ‘subject’ includes mammals, including non-human animals and especially humans. In one embodiment the patient or subject is a human. In another embodiment the patient or subject is a human male. In another embodiment the patient or subject is a human female.

The term ‘significant’ or ‘significantly’ is determined by t-test at 0.05 level of significance.

The present invention relates to methods of using of 6-methyl-5,6,6a,7-tetrahydro-4H-dibenzo[de,g]quinoline-10,11-diol to activate HSF1, to activate the HSF1 pathway, to increase the level of transcription of genes positively regulated by HSF1, to increase the level of protein chaperones and/or co-chaperones, to reduce the amount of protein misfolding, or to reduce the accumulation of misfolded proteins in a cell, tissue or animal.

The present invention further relates to methods of using 6-methyl-5,6,6a,7-tetrahydro-4H-dibenzo[de,g]quinoline-10,11-diol; for the treatment of a disease or disorder that is mediated by protein misfolding or accumulation of misfolded proteins; or for preventing, alleviating, ameliorating, or reducing the risk of acquiring, a disease or disorder; by activating the HSF1 pathway. The present invention further relates to method of using 6-methyl-5,6,6a,7-tetrahydro-4H-dibenzo[de,g]quinoline-10,11-diol for extending/increasing the longevity of a cell, tissue, organ, or animal.

Accordingly, in one aspect, the present invention provides a method of activating HSF1 in a cell, comprising the step of contacting said cell with an effective amount of 6-methyl-5,6,6a,7-tetrahydro-4H-dibenzo[de,g]quinoline-10,11-diol.

In a related aspect, the present invention provides a method of increasing transcription of a gene that is transactivated by HSF1 in a cell, comprising the step of contacting said cell with an effective amount of 6-methyl-5,6,6a,7-tetrahydro-4H-dibenzo[de,g]quinoline-10,11-diol. In one embodiment, the gene encodes a protein selected from any one or more of: PPARGC1A (PGC1alpha), DLG4 (PSD95), SYN1 (Synapsin), BDNF, HSP70s, HSP40s (including Cysteine-string protein alpha, Auxillin), HSPA8 (HSC70), HSPB8, or BAG3.

In another aspect, the present invention provides a method of increasing protein chaperone and/or co-chaperone levels in a cell, comprising the step of contacting said cell with an effective amount of 6-methyl-5,6,6a,7-tetrahydro-4H-dibenzo[de,g]quinoline-10,11-diol. In one embodiment, said protein chaperone and/or co-chaperone is selected from any one or more of: HSP70s, HSP40s (including Cysteine-string protein alpha, Auxillin), HSPA8 (HSC70), HSPB8, or BAG3.

In another aspect, the present invention provides a method of: (a) reducing protein misfolding in a cell, in terms of frequency or rate at which protein misfolding occurs, (b) reducing accumulation of misfolded proteins in a cell, or (c) reducing protein aggregation in a cell, particularly aggregation of misfolded proteins, said method comprising the step of contacting said cell with an effective amount of 6-methyl-5,6,6a,7-tetrahydro-4H-dibenzo[de,g]quinoline-10,11-diol.

In another aspect, the invention provides a method of increasing cell lifespan, comprising the step of contacting said cell with an effective amount of 6-methyl-5,6,6a,7-tetrahydro-4H-dibenzo[de,g]quinoline-10,11-diol.

In one embodiment, the cell in one of the above aspects, or other aspect or embodiments herein, is a cell type or from a tissue selected from any one or more of: adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder and vagina. In a further embodiment, said brain cell is from a brain tissue selected from cerebrum (including cerebral cortex, basal ganglia (often called the striatum), and olfactory bulb), cerebellum (including dentate nucleus, interposed nucleus, fastigial nucleus, and vestibular nuclei), diencephalon (including thalamus, hypothalamus, etc. and the posterior portion of the pituitary gland), and brain-stem (including pons, substantia nigra, medulla oblongata). In a further embodiment, said brain cell is selected from a neuron or glia cell (e.g., an astrocyte, oligodendrocyte, or microglia). In a further embodiment, said neuron is a sensory neuron, motor neuron, interneuron, or brain neuron.

In one embodiment, the cell is an animal cell, e.g., mammalian cell. In a further embodiment, said cell in a human cell or non-human cell. In a further embodiment, said cell is a human cell. In a further embodiment, said cell is in vitro, in vivo, or ex vivo.

In another embodiment, the cell is a diseased cell. In another embodiment, the cell is diseased cell from a patient suffering from a disease or disorder disclosed herein.

In another aspect, the invention provides for a method of treating an animal having a disease or disorder: (a) with a symptom that is prevented, alleviated, or ameliorated by HSF1 activation; or, (b) with a disease process or progression that slowed, halted or reversed by HSF1 activation; the method comprising the step of administering a therapeutically effective amount of a pharmaceutical composition comprising 6-methyl-5,6,6a,7-tetrahydro-4H-dibenzo[de,g]quinoline-10,11-diol to said animal. In one embodiment, the animal is mammal. In a further embodiment, the mammal is a human. In another embodiment, the mammal is a non-human.

In another aspect, the invention provides for a method of: (a) treating an animal having a disease or disorder that would benefit from HSF1 activation; or (b) preventing or reducing the risk of acquiring said disease or disorder; the method comprising the step of administering a therapeutically effective amount of a pharmaceutical composition comprising 6-methyl-5,6,6a,7-tetrahydro-4H-dibenzo[de,g]quinoline-10,11-diol to said animal. In one embodiment, said animal is a mammal. In another embodiment, said mammal is a human or a non-human mammal. In another embodiment, said disease or disorder is caused by protein misfolding, accumulation of misfolded proteins, or protein aggregation. In another embodiment, said disease is selected from any one or more of: aging-related tau astrogliopathy (ARTA), Alexander Disease, Alpers-Huttenlocher syndrome, Alzheimer's disease, Amyotrophic Lateral Sclerosis (ALS), Ataxia neuropathy spectrum, ataxia and retinitis pigmentosa (NARP), Critical Illness Myopathy (CIM), Primary Age-Related Tauopathy (PART), aortic medial amyloidosis, ApoAI amyloidosis, ApoAII amyloidosis, ApoAIV amyloidosis, argyrophillic grain disease, ataxia telangiectasia, atrial fibrillation, Autosomal Dominant Hyper-IgE Syndrome, cardiac atrial amyloidosis, Bloom's syndrome, cardiovascular diseases (including coronary artery disease, myocardial infarction, stroke, restenosis and arteriosclerosis), cataracts, cerebral amyloid angiopathy, Christianson syndrome, chronic traumatic encephalopathy, Chronic progressive external ophthalmoplegia (CPEO), Cockayne's syndrome, congenital lactic acidosis (CLA), corneal lactoferrin amyloidosis, corticobasal degeneration, Crohn's Disease, Cushing's disease, cutaneous lichen amyloidosis, cystic fibrosis, Dentatorubropallidoluysian Atrophy (DRPLA), dialysis amyloidosis, diffuse neurofibrillary tangles with calcification, Down syndrome, endotoxin shock, familial amyloidosis of the Finnish type, familial amyloidotic neuropathy, Familial British Dementia (FBD), Familial Danish Dementia (FDD), familial dementia, fibrinogen amyloidosis, fragile X syndrome, Fragile X-associated Tremor/Ataxia Syndrome (FXTAS), Friedreich's ataxia, fronto-temporal degeneration, glaucoma, Glycogen Storage Disease type IV (Andersen Disease), Guadeloupean Parkinsonism, hereditary lattice corneal dystrophy, Huntington's disease, inclusion body myositis/myopathy, inflammation, inflammatory bowel disease, ischemic conditions (including ischemia/reperfusion injury, myocardial ischemia, stable angina, unstable angina, stroke, ischemic heart disease and cerebral ischemia), light chain or heavy chain amyloidosis, lysosomal storage diseases (including aspartylglucosaminuria, Fabry's disease, Batten disease, Cystinosis, Farber, Fucosidosis, Galactasidosialidosis, Gaucher's disease (including Types 1, 2 and 3), Gml gangliosidosis, Hunter's disease, Hurler-Scheie's disease, Krabbe's disease, a-Mannosidosis, Kearns-Sayre syndrome (KSS), lactic acidosis and stroke-like episodes (MELAS) syndrome, Leber hereditary optic neuropathy (LHON), B-Mannosidosis, Maroteaux-Lamy's disease, MEGDEL syndrome (also known as 3-methylglutaconic aciduria with deafness, encephalopathy and Leigh-like syndrome), Metachromatic Leukodystrophy, Mitochondrial neurogastro-intestinal encephalopathy (MNGIE) syndrome, Morquio A syndrome, Morquio B syndrome, Mucolipidosis II, Mucolipidosis III, Myoclonic epilepsy myopathy sensory ataxia, Mitochondrial myopathy, Myoclonic epilepsy with ragged red fibres (MERRF), Neimann-Pick Disease (including Types A, B and C), Neurogenic muscle weakness, Pearson syndrome, Pompe's disease, Sandhoff disease, Sanfilippo syndrome (including Types A, B, C and D), Schindler disease, Schindler-Kanzaki disease, Sengers syndrome, Sialidosis, Sly syndrome, Tay-Sach's disease, Wolman disease, lysozyme amyloidosis, Mallory bodies, medullary thyroid carcinoma, mitochondrial myopathies, multiple sclerosis, multiple system atrophy, myotonic dystrophy, myotonic dystrophy, neurodegeneration with brain iron accumulation, neurofibromatosis, neuronal ceroid lipofuscinosis, odontogenic (Pinborg) tumor amyloid, Parkinsonism-Dementia of Guam, Parkinson's disease, peptic ulcers, Pick's disease, pituitary prolactinoma, post-encephalitic Parkinsonism, prion diseases (also known as Transmissible Spongiform Encephalopathies or TSEs, including Creutzfeldt-Jakob Disease (CJD), Variant Creutzfeldt-Jakob Disease, Gerstmann-Straussler-Scheinker Syndrome, Fatal Familial Insomnia, and Kuru), progressive supranuclear palsy, pulmonary alveolar proteinosis, retinal ganglion cell degeneration in glaucoma, retinitis pigmentosa with rhodopsin mutations, seminal vesical amyloid, senile systemic amyloidoses, Serpinopathies, sickle cell disease, spinal and bulbar muscular atrophy (SBMA) (also known as Kennedy's disease), spinocerebellar ataxias (including spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 8, and spinocerebellar ataxia type 17), subacute sclerosing panencephalitis, tauopathies, type II diabetes, vascular dementia, Werner syndrome, atherosclerosis, autism spectrum disorder (ASD), benign focal amyotrophy, Duchenne's paralysis, hereditary spastic paraplegia (HSP), Kugelberg-Welander syndrome, Lou Gehrig's disease, necrotizing enterocolitis, Paget's disease of the bone (PDB), primary lateral sclerosis (PLS), progressive bulbar palsy (PBP), progressive muscular atrophy (PMA), pseudobulbar palsy, spinal muscular atrophy (SMA), ulcerative colitis, Valosin-Containing Protein (VCP)-related disorders, or Werdnig-Hoffmann disease, transient ischemic attack, ischaemia, cerebral hemorrhage, senile cataract, retinal ischemia, retinal vasculitis, Brown-Vialetto-Van Laere syndrome, Eales Disease, meningitis and encephalitis, post-traumatic stress disorder, Charcot-Marie-Tooth Disease, macular degeneration, X-Linked spino-bulbar muscular atrophy (Kennedy's disease), presenile dementia, depressive disorder, temporal lobe epilepsy, Hereditary Leber Optic Atrophy, cerebrovascular accident, subarachnoid hemorrhage, schizophrenia, demyelinating disorders, and Pelizaeus-Merzbacher disease.

In another embodiment, said disease is a neurological disease.

In one embodiment, the disease is selected from any one or more of: Lysosomal Storage Diseases (e.g., Niemann-Picks Type C, Gaucher Disease), inclusion body myositis, spinocerebellar ataxias, spinal and bulbar muscular atrophy, or a condition associated therewith.

In another embodiment, the disease is selected from one or more of: ALS, frontotemporal dementia, Huntington's disease, Alzheimer's disease, Parkinson's disease, dementia with Lewy bodies, Parkinson's disease dementia, neurodegeneration with brain iron accumulation, diffuse neurofibrillary tangles with calcification, multiple system atrophy, cerebral amyloid angiopathy, vascular dementia, Down's syndrome, Creutzfeldt-Jakob disease, fatal familial insomnia, Gerstmann-Straussler-Scheinker syndrome, kuru, familial British dementia, familial Danish dementia, Parkinsonism-Dementia of Guam, myotonic dystrophy, neuronal ceroid lipofuscinosis, or a condition associated therewith.

In another embodiment, the neurological disease is selected from any one or more of: Friedreich's ataxia, multiple sclerosis, mitochondrial myopathies, progressive supranuclear palsy, corticobasal degeneration, chronic traumatic encephalopathy, argyrophillic grain disease, subacute sclerosing panencephalitis, Christianson syndrome, post-encephalitic Parkinsonism, Guadeloupean Parkinsonism, aging-related tau astrogliopathy (ARTA), and primary age-related tauopathy (PART), Pick's disease, or a condition associated therewith.

In another aspect, the invention provides for a method of increasing lifespan or treating a disease or disorder resulting in accelerated aging or other abnormal aging process in an animal, the method comprising the step of administering a therapeutically effective amount of a pharmaceutical composition comprising 6-methyl-5,6,6a,7-tetrahydro-4H-dibenzo[de,g]quinoline-10,11-diol to said animal. In one embodiment, said animal is a mammal. In another embodiment, said mammal is a human or a non-human mammal. In one embodiment, said disease or disorder is selected from Werner syndrome, Hutchinson-Guilford disease, Bloom's syndrome, Cockayne's syndrome, ataxia telangiectasia, and Down syndrome.

In a related aspect, the invention provides for a method of treating premature aging due to chemical or radiation exposure. In one embodiment, the premature aging is due to exposure to chemotherapy, radiation therapy, or UV radiation. In a further embodiment, the UV radiation is artificial, e.g., tanning bed, or solar UV radiation, i.e., sun exposure.

Physical exercise results in muscle adaptations including muscle atrophy caused by muscle protein catabolism or muscle hypertrophy caused by muscle protein accretion. In muscle hypertrophy nascent proteins are formed. An increase in the presence of molecular chaperones will act to enhance the stability of these rapidly forming nascent proteins by preventing misfolding and catabolism. 6-methyl-5,6,6a,7-tetrahydro-4H-dibenzo[de,g]quinoline-10,11-diol can therefore be used to stabilize nascent proteins in situations of enhanced protein turnover, e.g., after physical exercise, by reducing misfolding and catabolism of nascent proteins. Accordingly, in another aspect, the present invention relates to a method of increasing muscle hypertrophy or reducing muscle atrophy in an animal following physical exercise, the method comprising the step of administering a therapeutically effective amount of a pharmaceutical composition comprising 6-methyl-5,6,6a,7-tetrahydro-4H-dibenzo[de,g]quinoline-10,11-diol to said animal.

The present invention further provides of the use of 6-methyl-5,6,6a,7-tetrahydro-4H-dibenzo[de,g]quinoline-10,11-diol for the preparation of a medicament for treating a human having any one of the diseases or disorders disclosed herein or for use in any method of the present invention involving the administration of 6-methyl-5,6,6a,7-tetrahydro-4H-dibenzo[de,g]quinoline-10,11-diol to a human.

In another aspect, the invention provides for an in vitro method of screening a candidate therapeutic agent(s) for its ability to activate the HSF1 pathway, the method comprising the steps of:
(a) exposing induced astrocytes derived from fibroblast stem cells to said candidate therapeutic;
(b) comparing amounts of misfolded SOD1 between said induced astrocytes exposed to said candidate therapeutic and control cells, e.g., induced astrocytes that are not exposed to said candidate therapeutic (i.e., unexposed induced astrocytes).

The present invention relates to methods of using of 6-methyl-5,6,6a,7-tetrahydro-4H-dibenzo[de,g]quinoline-10,11-diol to activate NRF2 or to activate the NRF2 pathway.

The invention further relates to methods of reducing oxidative stress in a cell, said method comprising the step of administering an effective amount of 6-methyl-5,6,6a,7-tetrahydro-4H-dibenzo[de,g]quinoline-10,11-diol to said cell.

In one aspect, the present invention provides for a method of activating NRF2 in a cell, comprising a step of contacting the cell with an effective amount of 6-methyl-5,6,6a,7-tetrahydro-4H-dibenzo[de,g]quinoline-10,11-diol.

In a related aspect, the present invention provides for a method of increasing transcription of a gene that is transactivated by NRF2 in a cell, comprising the step of contacting said cell with an effective amount of 6-methyl-5,6,6a,7-tetrahydro-4H-dibenzo[de,g]quinoline-10,11-diol.

In one embodiment, the cell in one of the above aspects, or other aspect herein, is a cell type or from a tissue selected from any one or more of: adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder and vagina. In a further embodiment, said brain cell is from a brain tissue selected from cerebrum (including cerebral cortex, basal ganglia (often called the striatum), and olfactory bulb), cerebellum (including dentate nucleus, interposed nucleus, fastigial nucleus, and vestibular nuclei), diencephalon (including thalamus, hypothalamus, etc. and the posterior portion of the pituitary gland), and brain-stem (including pons, substantia nigra, medulla oblongata). In a further embodiment, said brain cell is selected from a neuron or glia cell (e.g., an astrocyte, oligodendrocyte, or microglia). In a further embodiment, said neuron is a sensory neuron, motor neuron, interneuron, or brain neuron.

In one embodiment, the cell is an animal cell, e.g., mammalian cell. In a further embodiment, said cell in a human cell or non-human cell. In a further embodiment, said cell is in vitro, in vivo, or ex vivo.

In another embodiment, the cell is a diseased cell. In another embodiment, the cell is diseased cell from a patient suffering from a disease or disorder below.

In another aspect, the invention provides for a method of treating an animal having a disease or disorder that would benefit from increased NRF2 activation or combined HSF1 and NRF2 activation, or for preventing or reducing the risk of acquiring a disease or disorder in an animal, the method comprising a step of administering a therapeutically effective amount of a pharmaceutical composition comprising 6-methyl-5,6,6a,7-tetrahydro-4H-dibenzo[de,g]quinoline-10,11-diol to said animal. 6-Methyl-5,6,6a,7-tetrahydro-4H-dibenzo[de,g]quinoline-10,11-diol may further be used in methods for treating diseases or disorders in an animal mediated by increased oxidative stress or by reduced NRF2 activity, said method comprising a step of administering an effective amount of 6-methyl-5,6,6a,7-tetrahydro-4H-dibenzo[de,g]quinoline-10,11-diol to said animal. 6-Methyl-5,6,6a,7-tetrahydro-4H-dibenzo[de,g]quinoline-10,11-diol may further be used in methods for reducing inflammation or treating diseases or disorders mediated by inflammation in an animal, said method comprising a step of administering an effective amount of 6-methyl-5,6,6a,7-tetrahydro-4H-dibenzo[de,g]quinoline-10,11-diol to said animal.

In one embodiment, said animal is a mammal. In another embodiment, said mammal is a human or a non-human mammal. In a further embodiment, said mammal is a human. In another embodiment, said disease or disorder is selected from any one or more of: aging-related tau astrogliopathy (ARTA), ALS, Alzheimer's disease, argyrophillic grain disease, asthma, cerebral amyloid angiopathy, cerebral ischemia Christianson syndrome, chronic obstructive pulmonary disease, chronic traumatic encephalopathy, corticobasal degeneration, Creutzfeldt-Jakob disease, dementia with Lewy bodies, diffuse neurofibrillary tangles with calcification, Down's syndrome, emphysema, familial British dementia, familial Danish dementia, fatal familial insomnia, Friedreich's ataxia, frontotemporal dementia, Gerstmann-Straussler-Scheinker syndrome, Guadeloupean Parkinsonism, Huntington's disease, kuru, mitochondrial myopathies, multiple sclerosis, multiple system atrophy, myotonic dystrophy, neurodegeneration with brain iron accumulation, neuronal ceroid lipofuscinosis, Parkinson's disease dementia, Parkinson's disease, Parkinson's, Parkinsonism-Dementia of Guam, Pick's disease, post-encephalitic Parkinsonism, primary age-related tauopathy (PART), progressive supranuclear palsy, pulmonary fibrosis, sepsis, septic shock, subacute sclerosing panencephalitis, vascular dementia, or a condition associated therewith.

The pharmaceutical compositions of the present invention comprise a therapeutically effective amount 6-methyl-5,6,6a,7-tetrahydro-4H-dibenzo[de,g]quinoline-10,11-diol and at least one pharmaceutically acceptable excipient. The term “excipient” refers to a pharmaceutically acceptable, inactive substance used as a carrier for the pharmaceutically active ingredient (6-methyl-5,6,6a,7-tetrahydro-4H-dibenzo[de,g]quinoline-10,11-diol), and includes antiadherents, binders, coatings, disintegrants, fillers, diluents, solvents, flavors, bulkants, colours, glidants, dispersing agents, wetting agents, lubricants, preservatives, sorbents and sweeteners. The choice of excipient(s) will depend on factors such as the particular mode of administration and the nature of the dosage form. Solutions or suspensions used for injection or infusion can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, including autoinjectors, or multiple dose vials made of glass or plastic.

A pharmaceutical formulation of the present invention may be in any pharmaceutical dosage form. The pharmaceutical formulation may be, for example, a tablet, capsule, nanoparticulate material, e.g., granulated particulate material or a powder, a lyophilized material for reconstitution, liquid solution, suspension, emulsion or other liquid form, injectable suspension, solution, emulsion, etc., suppository, or topical or transdermal preparation or patch. The pharmaceutical formulations generally contain about 1% to about 99% by weight of 6-methyl-5,6,6a,7-tetrahydro-4H-dibenzo[de,g]quinoline-10,11-diol and 99% to 1% by weight of a suitable pharmaceutical excipient. In one embodiment, the dosage form is an oral dosage form. In another embodiment, the dosage form is a parenteral dosage form. In another embodiment, the dosage form is an enteral dosage form. In another embodiment, the dosage form is a topical dosage form. In one embodiment, the pharmaceutical dosage form is a unit dose. The term ‘unit dose’ refers to the amount of 6-methyl-5,6,6a,7-tetrahydro-4H-dibenzo[de,g]quinoline-10,11-diol administered to a patient in a single dose.

In some embodiments, a pharmaceutical composition of the present invention is delivered to a subject via a parenteral route, an enteral route, or a topical route.

Examples of parental routes the present invention include, without limitation, any one or more of the following: intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumoral, intratympanic, intrauterine, intravascular, intravenous (bolus or drip), intraventricular, intravesical, and/or subcutaneous.

Enteral routes of administration of the present invention include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories. Oral administration includes sublingual and buccal administration.

Topical administration includes administration to a body surface, such as skin or mucous membranes, including intranasal and pulmonary administration. Transdermal forms include cream, foam, gel, lotion or ointment. Intranasal and pulmonary forms include liquids and powders, e.g., liquid spray.

The dose may vary depending upon the dosage form employed, sensitivity of the patient, and the route of administration. Dosage and administration are adjusted to provide sufficient levels of the active agent(s) or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy.

In one embodiment, the daily dose of (6aS)-6-methyl-5,6,6a,7-tetrahydro-4H-dibenzo[de,g]quinoline-10,11-diol administered to a patient is selected from up to 200 mg, 175 mg, 150 mg, 125 mg, 100 mg, 90 mg, 80 mg, 70 mg, 60 mg, 50 mg, 30 mg, 25 mg, 20 mg, 15 mg, 14 mg, 13 mg, 12 mg, 11 mg, 10 mg, 9 mg, 8 mg, 7 mg, 6 mg, 5 mg, 4 mg, 3 mg, or up to 2 mg. In another embodiment, the daily dose is at least 1 mg, 2 mg, 3 mg, 4 mg, 5 mg, 6 mg, 7 mg, 8 mg, 9 mg, 10 mg, 12 mg, 13 mg, 14 mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1,000 mg, 2,000 mg, 3,000 mg, 4,000 mg, or at least 5,000 mg. In another embodiment, the daily dose is 1-2 mg, 2-4 mg, 1-5 mg, 5-7.5 mg, 7.5-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg, 50-75 mg, 75-100 mg, 100-125 mg, 125-150 mg, 150-175 mg, 175-200 mg, 5-200 mg, 5-300 mg, 5-400 mg, 5-500 mg, 5-600 mg, 5-700 mg, 5-800 mg, 5-900 mg, 5-1,000 mg, 5-2,000 mg, 5-5,000 mg or more than 5,000 mg.

In another embodiment, a single dose of 6-methyl-5,6,6a,7-tetrahydro-4H-dibenzo[de,g]quinoline-10,11-diol administered to a patient is selected from: 1 mg, 2 mg, 3 mg, 4 mg, 5 mg, 6 mg, 7 mg, 8 mg, 9 mg, 10 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 100 mg, 110 mg, 120 mg, 130 mg, 140 mg , 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg 490 mg, 500 mg, 600 mg, 700 mg, 800 mg, 900 mg, 1,000 mg, 2,000 mg, 3,000 mg, 4,000 mg, or 5,000 mg. In one embodiment, the single dose is administered by a route selected from any one of: oral, buccal, or sublingual administration. In another embodiment, said single dose is administered by injection, e.g., subcutaneous, intramuscular, or intravenous. In another embodiment, said single dose is administered by inhalation or intranasal administration.

As a non-limited example, the dose of 6-methyl-5,6,6a,7-tetrahydro-4H-dibenzo[de,g]quinoline-10,11-diol administered by subcutaneous injection may be about 3 to 50 mg per day to be administered in divided doses. A single dose of 6-methyl-5,6,6a,7-tetrahydro-4H-dibenzo[de,g]quinoline-10,11-diol administered by subcutaneous injection may be about 1-6 mg, preferably about 1-4 mg, 1-3 mg, or 2 mg. Other embodiments include ranges of about 5-5,000 mg, preferably about 100-1,000 mg, 100-500 mg, 200-400 mg, 250-350 mg, or 300 mg. Subcutaneous infusion may be preferable in those patients requiring division of injections into more than 10 doses daily. The continuous subcutaneous infusion dose may be 1 mg/hour daily and is generally increased according to response up to 4 mg/hour.

The fine particle dose of 6-methyl-5,6,6a,7-tetrahydro-4H-dibenzo[de,g]quinoline-10,11-diol administered by pulmonary administration, e.g., inhalation using a pressurized metered dose inhaler (pMDI), dry powder inhaler (DPI), soft-mist inhaler, nebulizer, or other device, may be in the range of about, 0.5-15 mg, preferably about 0.5-8 mg or 2-6 mg. Other embodiments include ranges of about 5-5,000 mg, preferably about 100-1,000 mg, 100-500 mg, 200-400 mg, 250-350 mg, or 300 mg. The Nominal Dose (ND), i.e., the amount of drug metered in the receptacle (also known as the Metered Dose), of 6-methyl-5,6,6a,7-tetrahydro-4H-dibenzo[de,g]quinoline-10,11-diol administered by pulmonary administration may be, for example, in the range of 0.5-15 mg, 3-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg. Other embodiments include ranges of about 5-5,000 mg, preferably about 100-1,000 mg, 100-500 mg, 200-400 mg, 250-350 mg, or 300 mg.

Long-acting pharmaceutical compositions may be administered, 1, 2, 3, 4, 5, 6, 7, 8, 9, 10 or more than 10 times daily (preferably ≤10 times per day), every other day, every 3 to 4 days, every week, or once every two weeks depending on half-life and clearance rate of the particular formulation.

In an embodiment of any of the above methods and compositions, 6-methyl-5,6,6a,7-tetrahydro-4H-dibenzo[de,g]quinoline-10,11-diol, 6-methyl-5,6,6a,7-tetrahydro-4H-dibenzo[de,g]quinoline-10,11-diol prodrug, or salt, solvates, hydrates, and co-crystals thereof is a racemic mixture of R and S enantiomers, or enriched in R enantiomer (i.e., the ratio of R to S enantiomer for all of 6-methyl-5,6,6a,7-tetrahydro-4H-dibenzo[de,g]quinoline-10,11-diol in the composition, or all 6-methyl-5,6,6a,7-tetrahydro-4H-dibenzo[de,g]quinoline-10,11-diol being administered, is from 5:1 to 1,000:1, from 10:1 to 10,000:1, or from 100:1 to 100,000:1, or over all 6-methyl-5,6,6a,7-tetrahydro-4H-dibenzo[de,g]quinoline-10,11-diol enantiomers in the composition is at least 98% R enantiomer, 99% enantiomer, 99.5% enantiomer, 99.9% enantiomer, or is free of any observable amount of S enantiomer), or enriched in S enantiomer (i.e., the ratio of S to R enantiomer for all of 6-methyl-5,6,6a,7-tetrahydro-4H-dibenzo[de,g]quinoline-10,11-diol in the composition, or all 6-methyl-5,6,6a,7-tetrahydro-4H-dibenzo[de,g]quinoline-10,11-diol being administered, is from 5:1 to 1,000:1, from 10:1 to 10,000:1, or from 100:1 to 100,000:1, or over all 6-methyl-5,6,6a,7-tetrahydro-4H-dibenzo[de,g]quinoline-10,11-diol enantiomers in the composition is at least 98% S enantiomer, 99% enantiomer, 99.5% enantiomer, 99.9% enantiomer, or is free of any observable amount of R enantiomer).

The present invention further provides an in vitro or ex vivo method of: activating HSF1 in a cell; increasing transcription of a gene that is transactivated by HSF1 in a cell; increasing protein chaperone and/or co-chaperone levels in a cell (such as one or more of HSP70s, HSP40s (including Cysteine-string protein alpha, Auxillin), HSPA8 (HSC70), HSPB8, or BAG3); or reducing protein misfolding, accumulation of misfolded protein, or aggregated protein in a cell, said method comprising the step of contacting said cell with an effective amount of 6-methyl-5,6,6a,7-tetrahydro-4H-dibenzo[de,g]quinoline-10,11-diol (such as (6aS)-6-methyl-5,6,6a,7-tetrahydro-4H-dibenzo[de,g]quinoline-10,11-diol).

Suitably, a misfolded protein or aggregated protein may be selected from any one of TDP-43, SOD1, hyperphosphorylated Tau, hexanucleotide repeat expansion C9orf72, β-amyloid, α-synuclein, polyglutamine repeat expansion, FUS, hnRNPs, ATXN2, or prion protein.

Suitably, in the methods of the invention the cell may a cell type or from a tissue selected from any one or more of: adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder or vagina. Suitably, a brain cell may be from a brain tissue selected from: cerebrum, cerebellum, diencephalon, or brain-stem. Suitably, a brain cell may be selected from: neuron (such as a sensory neuron, motor neuron, interneuron, or brain neuron), astrocyte, oligodendrocyte, or microglia.

Suitably, said cell may be an animal cell (such as a human cell).

Suitably, said cell is having a disease or disorder or at risk of said disease or disorder or at risk of acquiring said disease or disorder selected from any one or more of: aging-related tau astrogliopathy (ARTA), Alexander Disease, Alpers-Huttenlocher syndrome, Alzheimer's disease, Amyotrophic Lateral Sclerosis (ALS), Ataxia neuropathy spectrum, ataxia and retinitis pigmentosa (NARP), Critical Illness Myopathy (CIM), Primary Age-Related Tauopathy (PART), aortic medial amyloidosis, ApoAI amyloidosis, ApoAII amyloidosis, ApoAIV amyloidosis, argyrophillic grain disease, ataxia telangiectasia, atrial fibrillation, Autosomal Dominant Hyper-IgE Syndrome, cardiac atrial amyloidosis, Bloom's syndrome, cardiovascular diseases, coronary artery disease, myocardial infarction, stroke, restenosis, arteriosclerosis, cataracts, cerebral amyloid angiopathy, Christianson syndrome, chronic traumatic encephalopathy, Chronic progressive external opthalmoplegia (CPEO), Cockayne's syndrome, congenital lactic acidosis (CLA), corneal lactoferrin amyloidosis, corticobasal degeneration, Crohn's Disease, Cushing's disease, cutaneous lichen amyloidosis, cystic fibrosis, Dentatorubropallidoluysian Atrophy (DRPLA), dialysis amyloidosis, diffuse neurofibrillary tangles with calcification, Down syndrome, endotoxin shock, familial amyloidosis of the Finnish type, familial amyloidotic neuropathy, Familial British Dementia (FBD), Familial Danish Dementia (FDD), familial dementia, fibrinogen amyloidosis, fragile X syndrome, Fragile X-associated Tremor/Ataxia Syndrome (FXTAS), Friedreich's ataxia, fronto-temporal degeneration, glaucoma, Glycogen Storage Disease type IV (Andersen Disease), Guadeloupean Parkinsonism, hereditary lattice corneal dystrophy, Huntington's disease, inclusion body myositis/myopathy, inflammation, inflammatory bowel disease, ischemic condition, ischemia/reperfusion injury, myocardial ischemia, stable angina, unstable angina, stroke, ischemic heart disease and cerebral ischemia, light chain or heavy chain amyloidosis, lysosomal storage diseases, aspartylglucosaminuria, Fabry's disease, Batten disease, Cystinosis, Farber, Fucosidosis, Galactasidosialidosis, Gaucher's disease Type 1, 2 or 3, Gml gangliosidosis, Hunter's disease, Hurler-Scheie's disease, Krabbe's disease, a-Mannosidosis, Kearns-Sayre syndrome (KSS), lactic acidosis and stroke-like episodes (MELAS) syndrome, Leber hereditary optic neuropathy (LHON), B-Mannosidosis, Maroteaux-Lamy's disease, MEGDEL syndrome (also known as 3-methylglutaconic aciduria with deafness, encephalopathy and Leigh-like syndrome), Metachromatic Leukodystrophy, Mitochondrial neurogastro-intestinal encephalopathy (MNGIE) syndrome, Morquio A syndrome, Morquio B syndrome, Mucolipidosis II, Mucolipidosis III, Myoclonic epilepsy myopathy sensory ataxia, Mitochondrial myopathy, Myoclonic epilepsy with ragged red fibres (MERRF), Neimann-Pick Disease Type A, B or C, Neurogenic muscle weakness, Pearson syndrome, Pompe's disease, Sandhoff disease, Sanfilippo syndrome Type A, B, C or D, Schindler disease, Schindler-Kanzaki disease, Sengers syndrome, Sialidosis, Sly syndrome, Tay-Sach's disease, Wolman disease, lysozyme amyloidosis, Mallory bodies, medullary thyroid carcinoma, mitochondrial myopathies, multiple sclerosis, multiple system atrophy, myotonic dystrophy, myotonic dystrophy, neurodegeneration with brain iron accumulation, neurofibromatosis, neuronal ceroid lipofuscinosis, odontogenic (Pinborg) tumor amyloid, Parkinsonism-Dementia of Guam, Parkinson's disease, peptic ulcers, Pick's disease, pituitary prolactinoma, post-encephalitic Parkinsonism, prion diseases (Transmissible Spongiform Encephalopathies), including Creutzfeldt-Jakob Disease (CJD), Variant Creutzfeldt-Jakob Disease, Gerstmann-Straussler-Scheinker Syndrome, Fatal Familial Insomnia, Kuru, progressive supranuclear palsy, pulmonary alveolar proteinosis, retinal ganglion cell degeneration in glaucoma, retinitis pigmentosa with rhodopsin mutations, seminal vesical amyloid, senile systemic amyloidoses, Serpinopathies, sickle cell disease, spinal and bulbar muscular atrophy (SBMA), spinocerebellar ataxias, spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 8, spinocerebellar ataxia type 17), subacute sclerosing panencephalitis, tauopathies, type II diabetes, vascular dementia, Werner syndrome, atherosclerosis, autism spectrum disorder (ASD), benign focal amyotrophy, Duchenne's paralysis, hereditary spastic paraplegia (HSP), Kugelberg-Welander syndrome, Lou Gehrig's disease, necrotizing enterocolitis, Paget's disease of the bone (PDB), primary lateral sclerosis (PLS), progressive bulbar palsy (PBP), progressive muscular atrophy (PMA), pseudobulbar palsy, spinal muscular atrophy (SMA), ulcerative colitis, Valosin-Containing Protein (VCP)-related disorders, or Werdnig-Hoffmann disease, transient ischemic attack, ischaemia, cerebral hemorrhage, senile cataract, retinal ischemia, retinal vasculitis, Brown-Vialetto-Van Laere syndrome, Eales Disease, meningitis and encephalitis, post-traumatic stress disorder, Charcot-Marie-Tooth Disease, macular degeneration, X-Linked spino-bulbar muscular atrophy (Kennedy's disease), presenile dementia, depressive disorder, temporal lobe epilepsy, Hereditary Leber Optic Atrophy, cerebrovascular accident, subarachnoid hemorrhage, schizophrenia, demyelinating disorders, and Pelizaeus-Merzbacher disease.

The present invention also relates to a therapeutically effective amount of 6-methyl-5,6,6a,7-tetrahydro-4H-dibenzo[de,g]quinoline-10,11-diol (or a composition comprising 6-methyl-5,6,6a,7-tetrahydro-4H-dibenzo[de,g]quinoline-10,11-diol) for use in: 1) treating an animal having a disease or disorder that would benefit from increased HSF1 activation in a subject; 2) preventing or reducing the risk of acquiring a disease or disorder in a subject by increasing HSF1 activation and/or 3) increasing muscle hypertrophy or reducing muscle atrophy in an animal following physical exercise. Suitably, the disease or disorder may be selected from any one or more of: aging-related tau astrogliopathy (ARTA), Alpers-Huttenlocher syndrome, Alexander Disease, Alzheimer's disease, Amyotrophic Lateral Sclerosis (ALS), Ataxia neuropathy spectrum, ataxia and retinitis pigmentosa (NARP), Critical Illness Myopathy (CIM), Primary Age-Related Tauopathy (PART), aortic medial amyloidosis, ApoAI amyloidosis, ApoAII amyloidosis, ApoAIV amyloidosis, argyrophillic grain disease, ataxia telangiectasia, atrial fibrillation, Autosomal Dominant Hyper-IgE Syndrome, cardiac atrial amyloidosis, Bloom's syndrome, cardiovascular diseases, coronary artery disease, myocardial infarction, stroke, restenosis, arteriosclerosis, cataracts, cerebral amyloid angiopathy, Christianson syndrome, chronic traumatic encephalopathy, Chronic progressive external ophthalmoplegia (CPEO), Cockayne's syndrome, congenital lactic acidosis (CLA), corneal lactoferrin amyloidosis, corticobasal degeneration, Crohn's Disease, Cushing's disease, cutaneous lichen amyloidosis, cystic fibrosis, Dentatorubropallidoluysian Atrophy (DRPLA), dialysis amyloidosis, diffuse neurofibrillary tangles with calcification, Down syndrome, endotoxin shock, familial amyloidosis of the Finnish type, familial amyloidotic neuropathy, Familial British Dementia (FBD), Familial Danish Dementia (FDD), familial dementia, fibrinogen amyloidosis, fragile X syndrome, Fragile X-associated Tremor/Ataxia Syndrome (FXTAS), Friedreich's ataxia, fronto-temporal degeneration, glaucoma, Glycogen Storage Disease type IV (Andersen Disease), Guadeloupean Parkinsonism, hereditary lattice corneal dystrophy, Huntington's disease, inclusion body myositis/myopathy, inflammation, inflammatory bowel disease, ischemic condition, ischemia/reperfusion injury, myocardial ischemia, stable angina, unstable angina, stroke, ischemic heart disease and cerebral ischemia, light chain or heavy chain amyloidosis, lysosomal storage diseases, aspartylglucosaminuria, Fabry's disease, Batten disease, Cystinosis, Farber, Fucosidosis, Galactasidosialidosis, Gaucher's disease Type 1, 2 or 3, Gml gangliosidosis, Hunter's disease, Hurler-Scheie's disease, Krabbe's disease, a-Mannosidosis, Kearns-Sayre syndrome (KSS), lactic acidosis and stroke-like episodes (MELAS) syndrome, Leber hereditary optic neuropathy (LHON), B-Mannosidosis, Maroteaux-Lamy's disease, MEGDEL syndrome (also known as 3-methylglutaconic aciduria with deafness, encephalopathy and Leigh-like syndrome), Metachromatic Leukodystrophy, Mitochondrial neurogastro-intestinal encephalopathy (MNGIE) syndrome, Morquio A syndrome, Morquio B syndrome, Mucolipidosis II, Mucolipidosis III, Myoclonic epilepsy myopathy sensory ataxia, Mitochondrial myopathy, Myoclonic epilepsy with ragged red fibres (MERRF), Neimann-Pick Disease Type A, B or C, Neurogenic muscle weakness, Pearson syndrome, Pompe's disease, Sandhoff disease, Sanfilippo syndrome Type A, B, C or D, Schindler disease, Schindler-Kanzaki disease, Sengers syndrome, Sialidosis, Sly syndrome, Tay-Sach's disease, Wolman disease, lysozyme amyloidosis, Mallory bodies, medullary thyroid carcinoma, mitochondrial myopathies, multiple sclerosis, multiple system atrophy, myotonic dystrophy, myotonic dystrophy, neurodegeneration with brain iron accumulation, neurofibromatosis, neuronal ceroid lipofuscinosis, odontogenic (Pinborg) tumor amyloid, Parkinsonism-Dementia of Guam, Parkinson's disease, peptic ulcers, Pick's disease, pituitary prolactinoma, post-encephalitic Parkinsonism, prion diseases (Transmissible Spongiform Encephalopathies), including Creutzfeldt-Jakob Disease (CJD), Variant Creutzfeldt-Jakob Disease, Gerstmann-Straussler-Scheinker Syndrome, Fatal Familial Insomnia, Kuru, progressive supranuclear palsy, pulmonary alveolar proteinosis, retinal ganglion cell degeneration in glaucoma, retinitis pigmentosa with rhodopsin mutations, seminal vesical amyloid, senile systemic amyloidoses, Serpinopathies, sickle cell disease, spinal and bulbar muscular atrophy (SBMA), spinocerebellar ataxias, spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 8, spinocerebellar ataxia type 17), subacute sclerosing panencephalitis, tauopathies, type II diabetes, vascular dementia, Werner syndrome, atherosclerosis, autism spectrum disorder (ASD), benign focal amyotrophy, Duchenne's paralysis, hereditary spastic paraplegia (HSP), Kugelberg-Welander syndrome, Lou Gehrig's disease, necrotizing enterocolitis, Paget's disease of the bone (PDB), primary lateral sclerosis (PLS), progressive bulbar palsy (PBP), progressive muscular atrophy (PMA), pseudobulbar palsy, spinal muscular atrophy (SMA), ulcerative colitis, Valosin-Containing Protein (VCP)-related disorders, or Werdnig-Hoffmann disease, transient ischemic attack, ischaemia, cerebral hemorrhage, senile cataract, retinal ischemia, retinal vasculitis, Brown-Vialetto-Van Laere syndrome, Eales Disease, meningitis and encephalitis, post-traumatic stress disorder, Charcot-Marie-Tooth Disease, macular degeneration, X-Linked spino-bulbar muscular atrophy (Kennedy's disease), presenile dementia, depressive disorder, temporal lobe epilepsy, Hereditary Leber Optic Atrophy, cerebrovascular accident, subarachnoid hemorrhage, schizophrenia, demyelinating disorders, and Pelizaeus-Merzbacher disease.

Suitably, the disease or disorder may be selected from any one or more of: Lysosomal Storage Disease, inclusion body myositis, spinocerebellar ataxias, or spinal and bulbar muscular atrophy.

Suitably, Lysosomal Storage Disease may be selected from Niemann-Picks Type C or Gaucher Disease.

Suitably, the disease or disorder may be selected from any one or more of: ALS, frontotemporal dementia, Huntington's disease, Alzheimer's disease, Parkinson's disease, dementia with Lewy bodies, Parkinson's disease dementia, neurodegeneration with brain iron accumulation, diffuse neurofibrillary tangles with calcification, multiple system atrophy, cerebral amyloid angiopathy, vascular dementia, Down's syndrome, Creutzfeldt-Jakob disease, fatal familial insomnia, Gerstmann-Straussler-Scheinker syndrome, kuru, familial British dementia, familial Danish dementia, Parkinsonism-Dementia of Guam, myotonic dystrophy, neuronal ceroid lipofuscinosis, or a condition associated therewith.

Suitably, the disease or disorder may be selected from any one or more of: Friedreich's ataxia, multiple sclerosis, mitochondrial myopathies, progressive supranuclear palsy, corticobasal degeneration, chronic traumatic encephalopathy, argyrophillic grain disease, subacute sclerosing panencephalitis, Christianson syndrome, aging-related tau astrogliopathy (ARTA), primary age-related tauopathy (PART), or Pick's disease.

Suitably, the subject or animal may be a mammal, such as a non-human animal or a human.

The present invention further provides the in vitro or ex vivo use of 6-methyl-5,6,6a,7-tetrahydro-4H-dibenzo[de,g]quinoline-10,11-diol (e.g. (6aS)-6-methyl-5,6,6a,7-tetrahydro-4H-dibenzo[de,g]quinoline-10,11-diol) to activate NRF2 in a cell or activate both HSF1 and NRF2 in a cell or reduce oxidative stress in a cell. Furthermore, the present invention provides in vitro or ex vivo methods of activating NRF2 in a cell or activating both HSF1 and NRF2 in a cell or reducing oxidative stress in a cell, comprising a step of contacting the cell with an effective amount of 6-methyl-5,6,6a,7-tetrahydro-4H-dibenzo[de,g]quinoline-10,11-diol (e.g. (6aS)-6-methyl-5,6,6a,7-tetrahydro-4H-dibenzo[de,g]quinoline-10,11-diol). Suitably, activation of NRF2 may comprise dissociation of NRF2 from Kelch-like ECH-associated protein.

Suitably, in uses and/or methods of the invention, the cell may be a cell type or from a tissue selected from any one or more of: adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder or vagina.

Suitably, the cell may be from an animal having a disease or disorder or at risk of acquiring said disease or disorder selected from any one or more of: aging-related tau astrogliopathy (ARTA), ALS, Alzheimer's disease, argyrophillic grain disease, asthma, cerebral amyloid angiopathy, cerebral ischemia Christianson syndrome, chronic obstructive pulmonary disease, chronic traumatic encephalopathy, corticobasal degeneration, Creutzfeldt-Jakob disease, dementia with Lewy bodies, diffuse neurofibrillary tangles with calcification, Down's syndrome, emphysema, familial British dementia, familial Danish dementia, fatal familial insomnia, Friedreich's ataxia, frontotemporal dementia, Gerstmann-Straussler-Scheinker syndrome, Guadeloupean Parkinsonism, Huntington's disease, kuru, mitochondrial myopathies, multiple sclerosis, multiple system atrophy, myotonic dystrophy, neurodegeneration with brain iron accumulation, neuronal ceroid lipofuscinosis, Parkinson's disease dementia, Parkinson's disease, Parkinson's, Parkinsonism-Dementia of Guam, Pick's disease, post-encephalitic Parkinsonism, primary age-related tauopathy (PART), progressive supranuclear palsy, pulmonary fibrosis, sepsis, septic shock, subacute sclerosing panencephalitis, vascular dementia, or a condition associated therewith.

The present invention also relates to a therapeutically effective amount of 6-methyl-5,6,6a,7-tetrahydro-4H-dibenzo[de,g]quinoline-10,11-diol (or a composition comprising 6-methyl-5,6,6a,7-tetrahydro-4H-dibenzo[de,g]quinoline-10,11-diol) for use in: 1) treating an animal having a disease or disorder that would benefit from increased NRF2 activation or that would benefit from a combination of increased HSF1 and increased NRF2 activation; or 2) preventing or reducing the risk of acquiring a disease or disorder in an animal by increasing NRF2 activation or by increasing both HSF1 and NRF2 activation. Suitably, said disease or disorder may be selected from any one or more of: aging-related tau astrogliopathy (ARTA), ALS, Alzheimer's disease, argyrophillic grain disease, asthma, cerebral amyloid angiopathy, cerebral ischemia Christianson syndrome, chronic obstructive pulmonary disease, chronic traumatic encephalopathy, corticobasal degeneration, Creutzfeldt-Jakob disease, dementia with Lewy bodies, diffuse neurofibrillary tangles with calcification, Down's syndrome, emphysema, familial British dementia, familial Danish dementia, fatal familial insomnia, Friedreich's ataxia, frontotemporal dementia, Gerstmann-Straussler-Scheinker syndrome, Guadeloupean Parkinsonism, Huntington's disease, kuru, mitochondrial myopathies, multiple sclerosis, multiple system atrophy, myotonic dystrophy, neurodegeneration with brain iron accumulation, neuronal ceroid lipofuscinosis, Parkinson's disease dementia, Parkinson's disease, Parkinson's, Parkinsonism-Dementia of Guam, Pick's disease, post-encephalitic Parkinsonism, primary age-related tauopathy (PART), progressive supranuclear palsy, pulmonary fibrosis, sepsis, septic shock, subacute sclerosing panencephalitis, vascular dementia, or a condition associated therewith.

Suitably, said animal may be a mammal, such as a non-human mammal or a human.

Suitably, in the methods, composition and/or second medical uses of the present invention, 6-methyl-5,6,6a,7-tetrahydro-4H-dibenzo[de,g]quinoline-10,11-diol (such as (6aS)-6-methyl-5,6,6a,7-tetrahydro-4H-dibenzo[de,g]quinoline-10,11-diol) may be administered or formulated for administration at a dose of 0.12 mg/kg or higher. Suitably, 6-methyl-5,6,6a,7-tetrahydro-4H-dibenzo[de,g]quinoline-10,11-diol may be administered at a dose between 10-5000 mg/day.

Suitably, in the methods, composition and/or second medical uses of the present invention, 6-methyl-5,6,6a,7-tetrahydro-4H-dibenzo[de,g]quinoline-10,11-diol (such as (6aS)-6-methyl-5,6,6a,7-tetrahydro-4H-dibenzo[de,g]quinoline-10,11-diol) may be administered or formulated for administration in any suitable way, for example parenterally, enterally, or topically.

Suitably, in the methods, composition and/or second medical uses of the present invention, 6-methyl-5,6,6a,7-tetrahydro-4H-dibenzo[de,g]quinoline-10,11-diol (such as (6aS)-6-methyl-5,6,6a,7-tetrahydro-4H-dibenzo[de,g]quinoline-10,11-diol) may be administered or formulated for administration by oral, sublingual, buccal, pulmonary, intranasal, intravenous, intramuscular or subcutaneous administration.

Another embodiment of the present invention includes use of 6-methyl-5,6,6a,7-tetrahydro-4H-dibenzo[de,g]quinoline-10,11-diol to slow down the decline of CMAP, or improve the CMAP. Alternatively, 6-methyl-5,6,6a,7-tetrahydro-4H-dibenzo[de,g]quinoline-10,11-diol may be for use in slowing down the decline of CMAP, or improving the CMAP, optionally for use in the treatment of a disease or disorder by slowing down the decline of CMAP, or improving the CMAP.

Another embodiment of the present invention includes use of 6-methyl-5,6,6a,7-tetrahydro-4H-dibenzo[de,g]quinoline-10,11-diol to improve the muscle strength. Alternatively, 6-methyl-5,6,6a,7-tetrahydro-4H-dibenzo[de,g]quinoline-10,11-diol may be for use in improving muscle strength, optionally for use in the treatment of a disease or disorder by improving muscle strength.

Another embodiment of the present invention includes use of 6-methyl-5,6,6a,7-tetrahydro-4H-dibenzo[de,g]quinoline-10,11-diol to control the body weight during the treatment of frontotemporal dementia. Alternatively, 6-methyl-5,6,6a,7-tetrahydro-4H-dibenzo[de,g]quinoline-10,11-diol may be for use in controlling body weight, optionally during the treatment of frontotemporal dementia, or in a patient having frontotemporal dementia.

One embodiment includes use of 6-methyl-5,6,6a,7-tetrahydro-4H-dibenzo[de,g]quinoline-10,11-diol to increase expression of heat shock protein Hspa8. Alternatively, 6-methyl-5,6,6a,7-tetrahydro-4H-dibenzo[de,g]quinoline-10,11-diol may be for use in increasing expression of heat shock protein Hspa8, optionally for use in the treatment of a disease or disorder by increasing expression of heat shock protein Hspa8.

Another embodiment includes use of 6-methyl-5,6,6a,7-tetrahydro-4H-dibenzo[de,g]quinoline-10,11-diol to increase expression of heat shock protein Hspa1a. Alternatively, 6-methyl-5,6,6a,7-tetrahydro-4H-dibenzo[de,g]quinoline-10,11-diol may be for use in increasing expression of heat shock protein Hspa1a, optionally for use in the treatment of a disease or disorder by increasing expression of heat shock protein Hspa1a.

Yet another embodiment includes use of 6-methyl-5,6,6a,7-tetrahydro-4H-dibenzo[de,g]quinoline-10,11-diol for the preparation of a medicament for increasing heat shock protein Hspa8 or Hspa1a.

Another embodiment includes use of 6-methyl-5,6,6a,7-tetrahydro-4H-dibenzo[de,g]quinoline-10,11-diol for the preparation of an orally administered medicament for increasing heat shock protein Hspa8 or Hspa1a.

### EXAMPLES

The present invention is not to be limited in scope by the specific embodiments described herein. Indeed various modifications of the invention in addition to those described herein will be apparent to those skilled in the art from the foregoing description and the accompanying Figures. Such modifications are intended to fall within the scope of the appended claims.

It is further to be understood that all values are approximate and are provided for description. All references cited and discussed in this specification are incorporated herein by reference in their entirety and to the same extent as if each reference was individually incorporated by reference.

The ability of (6aS)-6-methyl-5,6,6a,7-tetrahydro-4H-dibenzo[de,g]quinoline-10,11-diol to induce HSF1-regulated gene expression in the central nervous system was evaluated in the preclinical in vivo model.

### Example 1: In Vivo Pharmacodynamic Study Via Subcutaneous Dosing

Methodology

Wildtype mice (3 per group) were dosed subcutaneously at 0, 0.5, 1.5, 5 and 10 mg/kg (6aS)-6-methyl-5,6,6a,7-tetrahydro-4H-dibenzo[de,g]quinoline-10,11-diol hydrochloride for 7 days once daily. One animal in the 10 mg/kg dosing group was actually dosed 15 mg/kg on Day 1. To prepare the dosing solutions, (6aS)-6-methyl-5,6,6a,7-tetrahydro-4H-dibenzo[de,g]quinoline-10,11-diol was pre-weighed into bijous and stored in foil. Each dose was made up fresh daily with filter sterilized vehicle (0.9% (w/v) saline, 0.05% (w/v) sodium metabisulfite, and 1% (w/v) ascorbic acid, at pH 3.5. Tissues were collected at expected peak mRNA expression (6 hours post final dose) and expected trough expression (24 hours post final dose). During tissue collection, spinal cord and cortex were collected from each animal. RNA was extracted immediately using Rneasy® lipid tissue mini kit. RNA was treated with DNase and converted to cDNA. Measurement of gene expressions included Pgc1a, Dnajb1, Hspa1a, which are upregulated by HSF1 activation. Measurement of gene expressions included Gclm and Nqo1, which are upregulated by NRF2 activation.

Result:

Induction of gene expression result by (6aS)-6-methyl-5,6,6a,7-tetrahydro-4H-dibenzo[de,g]quinoline-10,11-diol was evaluated in wildtype mouse model. At 6 hours post final dose compared to Gapdh, 10 mg/kg (6aS)-6-methyl-5,6,6a,7-tetrahydro-4H-dibenzo[de,g]quinoline-10,11-diol exhibited a 2.2-fold Hspa1a gene induction and 1.3-fold Dnajb1 gene induction. The repeated dose of 5 mg/kg (6aS)-6-methyl-5,6,6a,7-tetrahydro-4H-dibenzo[de,g]quinoline-10,11-diol also gave a 1.8-fold Pgc1a gene induction. At 24 hours post final dose compared to Gapdh, 5 mg/kg (6aS)-6-methyl-5,6,6a,7-tetrahydro-4H-dibenzo[de,g]quinoline-10,11-diol exhibited a 2.3-fold Hspa1a gene induction and 1.7-fold Pgc1a gene induction.

At 6 hours post final dose compared to Actb, 10 mg/kg (6aS)-6-methyl-5,6,6a,7-tetrahydro-4H-dibenzo[de,g]quinoline-10,11-diol showed a 2.0-fold Hspa1a gene induction and 1.5-fold Pgc1a gene induction. At 24 hours post final dose compared to Actb, 1.5 mg/kg (6aS)-6-methyl-5,6,6a,7-tetrahydro-4H-dibenzo[de,g]quinoline-10,11-diol exhibited a 2.3-fold Hspa1a gene induction and 1.7-fold Pgc1a gene induction.

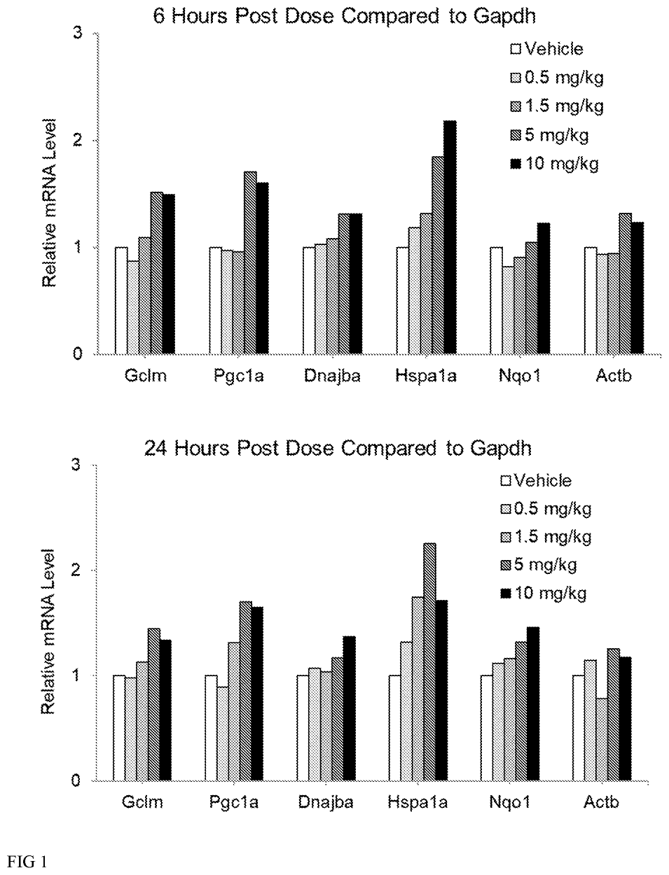

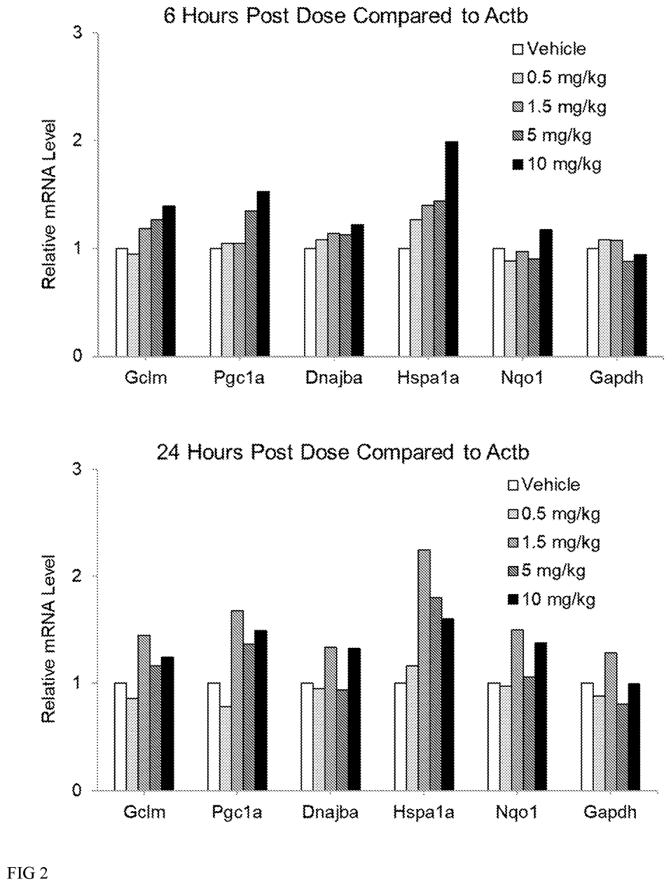

The result of Hspa1a and Dnajb1 gene inductions by (6aS)-6-methyl-5,6,6a,7-tetrahydro-4H-dibenzo[de,g]quinoline-10,11-diol has indicated that (6aS)-6-methyl-5,6,6a,7-tetrahydro-4H-dibenzo[de,g]quinoline-10,11-diol can activate HSF1 regulated genes at 0.5 mg/kg and above in mice (FIGS. 1 and 2).

Gene expressions including Gclm and Nqo1 that are regulated by NRF2 activation were also measured, as show in FIGS. 1 and 2. At 6 hours post final dose compared to Gapdh, 5 mg/kg (6aS)-6-methyl-5,6,6a,7-tetrahydro-4H-dibenzo[de,g]quinoline-10,11-diol exhibited a 1.5-fold Gclm gene induction. At 24 hours post final-dose compared to Gapdh, 5 mg/kg (6aS)-6-methyl-5,6,6a,7-tetrahydro-4H-dibenzo[de,g]quinoline-10,11-diol exhibited a 1.5-fold Gclm gene induction and 1.3-fold Nqo1 gene induction.

At 6 hours post final-dose compared to Actb, 10 mg/kg (6aS)-6-methyl-5,6,6a,7-tetrahydro-4H-dibenzo[de,g]quinoline-10,11-diol showed a 1.4-fold Gclm gene induction. At 24 hours post final-dose compared to Actb, 10 mg/kg (6aS)-6-methyl-5,6,6a,7-tetrahydro-4H-dibenzo[de,g]quinoline-10,11-diol exhibited a 1.2-fold Gclm gene induction and 1.4-fold 1.4 gene induction.

### Example 2: In Vivo Pharmacodynamic Study Via Subcutaneous Dosing

Methodology

Wildtype mice (3 per group) were dosed subcutaneously at 0, 0.5, 1.5, 5 and 10 mg/kg (6aS)-6-methyl-5,6,6a,7-tetrahydro-4H-dibenzo[de,g]quinoline-10,11-diol hydrochloride for 7 days once daily. One animal in the 10 mg/kg dosing group was actually dosed 15 mg/kg on Day 1. To prepare the dosing solutions, (6aS)-6-methyl-5,6,6a,7-tetrahydro-4H-dibenzo[de,g]quinoline-10,11-diol was pre-weighed into bijous and stored in foil. Each dose was made up fresh daily with filter sterilized vehicle (0.9% (w/v) saline, 0.05% (w/v) sodium metabisulfite, and 1% (w/v) ascorbic acid, at pH 3.5. Tissues were collected at expected peak mRNA expression (6 hours post final dose) and expected trough expression (24 hours post final dose). During tissue collection, spinal cord and cortex were collected from each animal. RNA was extracted immediately using Rneasy® lipid tissue mini kit. RNA was treated with DNase and converted to cDNA. Measurement of gene expressions included Hspa1a, Hspa8, Dlg4, Syn1, and Dnajb1, which are upregulated by HSF1 activation. Measurement of gene expressions included Gclm, Hmox1, Nqo1 and Pgc1a, which are upregulated by NRF2 activation.

Result

Induction of gene expression result by (6aS)-6-methyl-5,6,6a,7-tetrahydro-4H-dibenzo[de,g]quinoline-10,11-diol was evaluated in wildtype mouse model.

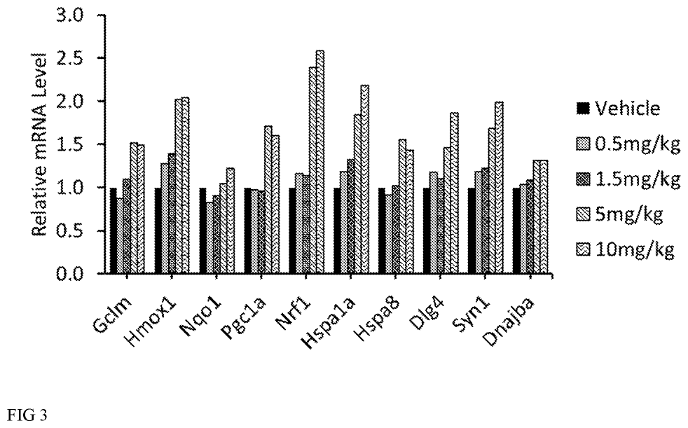

At 6 hours post final dose compared to Gapdh, 5 mg/kg (6aS)-6-methyl-5,6,6a,7-tetrahydro-4H-dibenzo[de,g]quinoline-10,11-diol exhibited an Hspa1a gene induction of 1.84-fold, an Hspa8 gene induction of 1.56-fold, a Dlg4 gene induction of 1.46-fold, a Syn1 gene induction of 1.68-fold, and a Dnajba gene induction of 1.31-fold, as shown in FIG. 3.

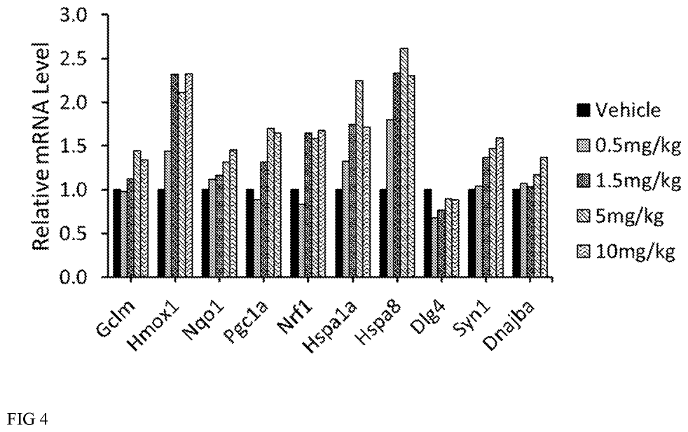

At 24 hours post final dose compared to Gapdh, 5 mg/kg (6aS)-6-methyl-5,6,6a,7-tetrahydro-4H-dibenzo[de,g]quinoline-10,11-diol exhibited an Hspa1a gene induction of 2.25-fold, an Hspa8 gene induction of 2.62-fold, a Dlg4 gene induction of 0.90-fold, a Syn1 gene induction of 1.47-fold, and a Dnajba gene induction of 1.17-fold, as shown in FIG. 4.

Gene expressions including Gclm and Nqo1 that are regulated by NRF2 activation were also measured, as show in FIGS. 3 and 4.

At 6 hours post final dose compared to Gapdh, 5 mg/kg (6aS)-6-methyl-5,6,6a,7-tetrahydro-4H-dibenzo[de,g]quinoline-10,11-diol exhibited a Gclm gene induction of 1.51-fold, an Hmox1 gene induction of 2.02-fold, an Nqo1 gene induction of 1.04-fold, a Pgc1a gene induction of 1.71-fold, and an Nrf1 gene induction of 2.39-fold, as shown in FIG. 3.

At 24 hours post final dose compared to Gapdh, 5 mg/kg (6aS)-6-methyl-5,6,6a,7-tetrahydro-4H-dibenzo[de,g]quinoline-10,11-diol exhibited a Gclm gene induction of 1.45-fold, an Hmox1 gene induction of 2.11-fold, an Nqo1 gene induction of 1.32-fold, a Pgc1a gene induction of 1.70-fold, and an Nrf1 gene induction of 1.58-fold, as shown in FIG. 4.

The result of gene inductions has indicated that (6aS)-6-methyl-5,6,6a,7-tetrahydro-4H-dibenzo[de,g]quinoline-10,11-diol can activate HSF1 regulated genes at 0.5 mg/kg and above in mice.

### Example 3: In Vivo Pharmacodynamic Study Via Oral Dosing

In Vivo Study and Tissue Processing

Wildtype mice were dosed orally at 25 mg/kg (6aS)-6-methyl-5,6,6a,7-tetrahydro-4H-dibenzo[de,g]quinoline-10,11-diol for 4 days once daily.

A total of 36 mice were randomly divided into twelve groups. Each group had 3 mice. After oral administration of (6aS)-6-methyl-5,6,6a,7-tetrahydro-4H-dibenzo[de,g]quinoline-10,11-diol once a day for 4 consecutive days, brains of mice in each group were sampled individually at the time point of 0, 5, 15, 30 min, 1, 2, 3, 4, 8, 12, 24 and 48 hour post the last oral dose. A quarter of whole brain from each mouse in every time group underwent a real-time quantitative polymerase chain reaction (RT-qPCR) analysis. Before RNA extraction, brain tissues were frozen immediately in liquid nitrogen, and were transferred to −80° C. until further use.

RT-qPCR

Total RNA from 36 frozen brain samples were extracted using Trizol reagent (Invitrogen, Carlsbad, Calif., USA, 15596018) according to the manufacturer's instructions. After extraction, the integrity of total RNA was be checked by 1% agarose gel staining with ethidium bromide as well as by nanodrop (Thermo Fisher, USA). Then, 2 μg of total RNA was used for reverse transcription by M-MLV Reverse Transcriptase (Invitrogen, 28025-013) with gDNA removal (NEB, M0303S). qRT-PCR analysis was performed using LightCycler® 480 Probe Master (4887301001, Roche, Basel, Switzerland) and the Roche LightCycler480 (Roche). The data from triplicate experiments were subjected to statistical analysis and the target genes were normalized against the levels of actin beta (Actb) mRNA. The primers are listed as follows:

| Prime name | Sequence |
|---|---|
| Mus-Actb-taqman-F | TGGAATCCTGTGGCATCCAT |
| Mus-Actb-taqman-R | GCTAGGAGCCAGAGCAGTAA |
| Mus-Actb-probe | ACCACCAGACAGCACTGTGTTGGCA |
| Mus-Hspa1a-taqman-F | GCTGCTTCTCCTTGCGTTTA |
| Mus-Hspa1a-taqman-R | TGCTGTCACTTCACCTCCAA |
| Mus-Hspa1a-probe | AGTCCTACAGTGCAACCACCATGCA |
| Mus-Hspa8-taqman-F | TGGAACTATTGCTGGCCTCA |
| Mus-Hspa8-taqman-R | TTCCTTTCAGCTCCGACCTT |
| Mus-Hspa8-probe | ACTGCTGCTGCTATTGCTTACGGC |

Results

Test compound increases the gene expression of heat shock protein Hspa8 and Hspa1a

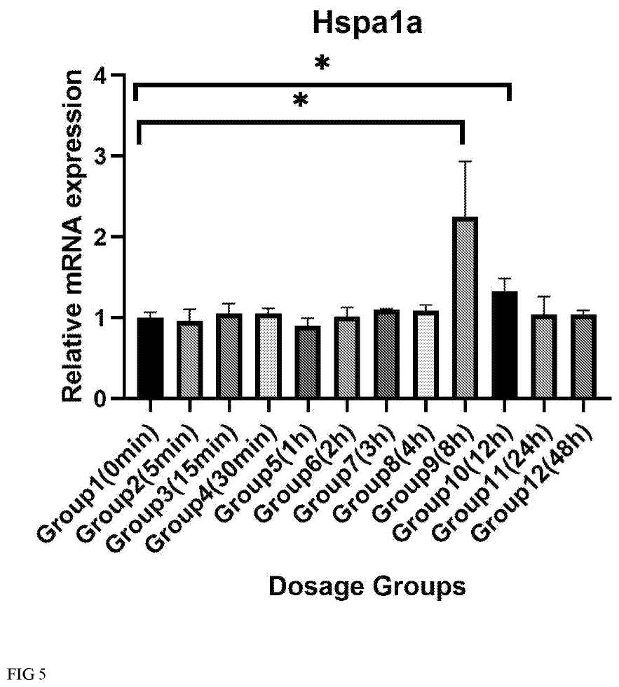

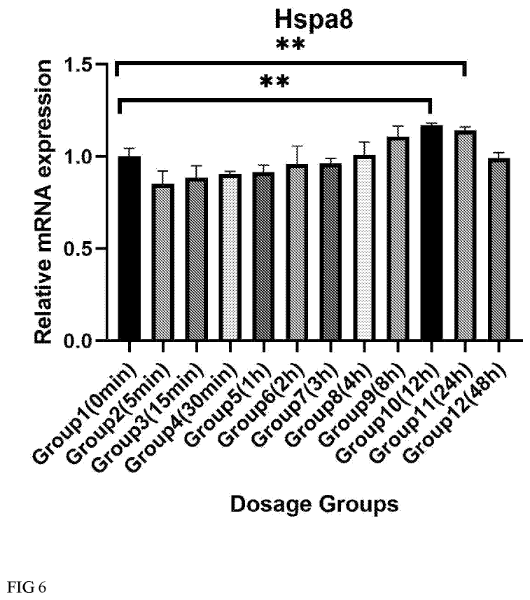

To evaluate the ability of the test compound to activate heat shock genes, RT-PCR was performed to detect the mRNA expression of Hspa8 and Hspa1a in brain samples. Our results show that the mRNA expression levels of Hspa8 exhibited a slight increase in group 10 and 11 compared with group 1. (Table 1). Similarly, Hspa1a mRNA level reached a peak in group 9 and showed a significant increase in group 9 and 10 compared with group 1 (Table 1., FIGS. 5 and 6).

TABLE 1
Relative expression of target genes
normalized with Group 1.

| | Relative expression | |
|---|---|---|
| | of target genes | |
| Groups | Hspa8 | Hspa1a |
| Group 1 (0 min) | 1.00 | 1.00 |
| Group 2 (5 min) | 0.85 | 0.96 |
| Group 3 (15 min) | 0.89 | 1.05 |
| Group 4 (30 min) | 0.91 | 1.06 |
| Group 5 (1 Hr) | 0.92 | 0.90 |
| Group 6 (2 Hr) | 0.96 | 1.02 |
| Group 7 (3 Hr) | 0.96 | 1.10 |
| Group 8 (4 Hr) | 1.01 | 1.09 |
| Group 9 (8 Hr) | 1.11 | 2.25* |
| Group 10 (12 Hr) | 1.17** | 1.33* |
| Group 11 (24 Hr) | 1.14** | 1.04 |
| Group 12 (48 Hr) | 0.99 | 1.04 |

*p < 0.5;
**p < 0.1

### Example 4: In Vivo Pharmacology Study in TDP-43^{Q331K} Mouse Model

Tg(Prnp-TARDBP*Q331K)103Dwc (also known as TDP-43^{Q331K}) transgenic mice have expression of a myc-tagged, human TAR DNA binding protein carrying the ALS-linked Q331K mutation (huTDP-43*Q331K) directed to brain and spinal cord by the mouse prion protein promoter. TDP-43^{Q331K} transgenic mice may be useful in studying motor dysfunction in the neurodegenerative disorder amyotrophic lateral sclerosis. The TDP-43^{Q331K} mouse model was initially imported from Jackson laboratories (USA) (Stock No: 017933) and characterized at the Sheffield Institute for Translational Neuroscience. All experiments involving mice were conducted in accordance with the animal (Scientific Procedures) Act 1986 and approved by the Sheffield University Ethical Review Committee Project Applications and Amendments Sub-Committee, and by the UK Animal Procedures Committee (London, UK).

The mouse colony was maintained in a specific pathogen free (SPF) environment before being moved for experiments to a conventional animal facility following the Home Office code of practice for the housing and care of animals used in scientific procedures.

Study Design

This in vivo pharmacology study was designed to test the efficacy of (6aS)-6-methyl-5,6,6a,7-tetrahydro-4H-dibenzo[de,g]quinoline-10,11-diol on the progression of motor and cognitive decline in the TDP-43^{Q331K} mouse model. In addition, tissue was collected at the end of the experiment for target engagement (gene expression) analysis of Nrf2 and HSF1 target genes.

Following genotyping, transgenic females were block randomized into three different dosing groups: vehicle once daily, 2.5 mg/kg (6aS)-6-methyl-5,6,6a,7-tetrahydro-4H-dibenzo[de,g]quinoline-10,11-diol twice daily and 5 mg/kg (6aS)-6-methyl-5,6,6a,7-tetrahydro-4H-dibenzo[de,g]quinoline-10,11-diol once daily. The doses were selected based on results of previous internal studies. Animals were weighed daily before dosing and dosed subcutaneously at 10 ml/kg with a solution of 0.5 mg/ml (6aS)-6-methyl-5,6,6a,7-tetrahydro-4H-dibenzo[de,g]quinoline-10,11-diol for the 5 mg/kg dose and 0.25 mg/ml for the 2.5 mg/kg dose. Where animals were dosed twice a day, the doses were at least 6 hours apart and the dosing solution was freshly made for the second dose of the day.

The study was designed with two main cohorts of mice. One cohort was dosed from 25 days of age until 6 months of age and mice had behavioral testing throughout the study and consisted of 14 mice per group. The other satellite cohort was dosed from 25 days of age until 3 months of age, with no behavioral testing and consisted of 6 mice per group for target engagement and histological assessment. Behavioral tests that were carried out during the experiment were: accelerated rotarod test, gait analysis, and electrophysiology.

Weighing

Animals were weighed daily before dosing to calculate dose volume. Animals that were dosed twice a day used the morning weight for the second dose.

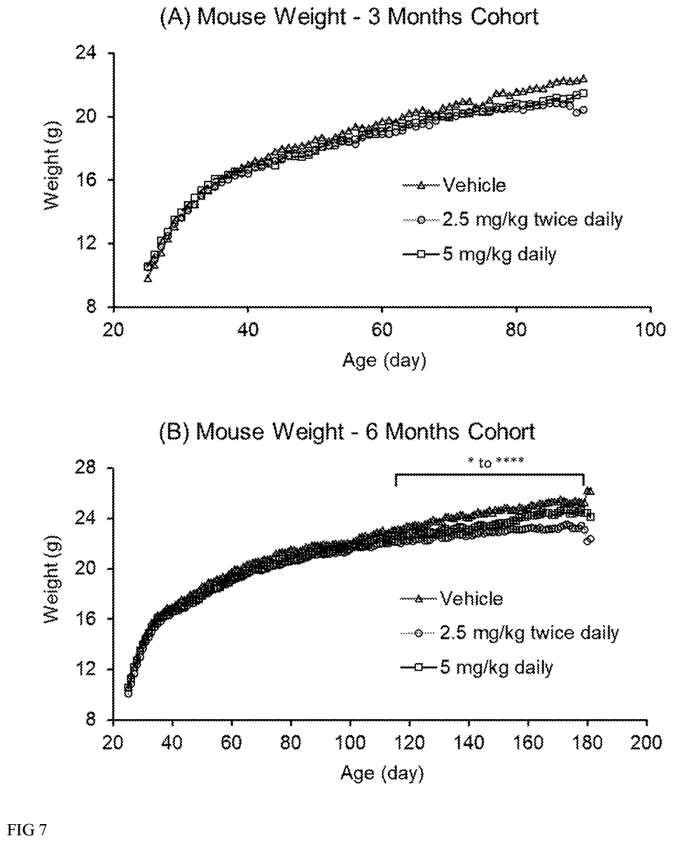

In the 3-month cohort, there was no significant difference in weight between either of the (6aS)-6-methyl-5,6,6a,7-tetrahydro-4H-dibenzo[de,g]quinoline-10,11-diol dosing groups compared to the vehicle dosing group (FIG. 7)

In the 6-month cohort there was a significant decrease in weight of the 2.5 mg/kg (6aS)-6-methyl-5,6,6a,7-tetrahydro-4H-dibenzo[de,g]quinoline-10,11-diol dosing group when compared to the vehicle dosing group from 121 days of age until the end of the study (FIG. 7).

Accelerating Rotarod Test

Mice were tested on the accelerating rotarod (Jones & Roberts for mice, model 7650) once a week from 40 days of age until the end of the study. The rotarod accelerates from 4 to 40 rpm over the course of 300 seconds. Mice are placed on the rotarod and the time taken to fall from the rotarod (latency to fall) is recorded. For each day of testing, each mouse was tested twice on the rotarod with a small rest in between trials and the best result from the two trials is recorded. The rotarod test was carried out at the same time of day each week (pm).

Before the first rotarod test, mice were trained on the rotarod for 3 consecutive days for two trials each. These results are recorded but are not used in analysis of the data.

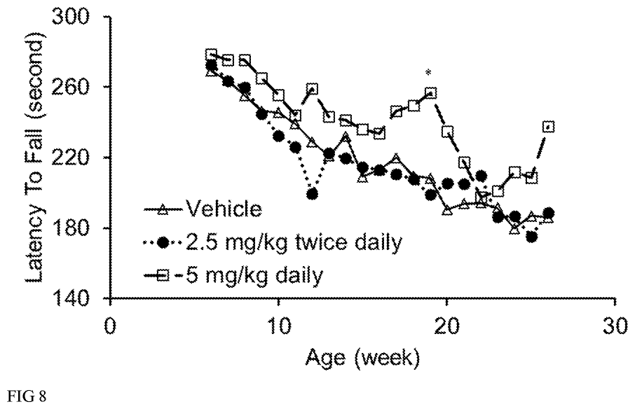

Rotarod performance consistently decreases in the TDP-43^{Q331K} animal model over time. There was a significant increase in the rotarod performance in the 5 mg/kg (6aS)-6-methyl-5,6,6a,7-tetrahydro-4H-dibenzo[de,g]quinoline-10,11-diol dosing group when compared to vehicle dosed animals at one time point (19 weeks of age). An increase in rotarod performance indicates an improved coordination and motor function (FIG. 8).

Catwalk Gait Analysis

Gait analysis was carried out at 3 months and 6 months of age using the catwalk gait analysis system 7.1 (Noldus Information Technology B.V., Netherlands) and analyzed using Catwalk software 7.1. The software calculated many different gait parameters such as stride length, base of support (BOS) and swing time as well as step patterns and percentage of time spent on 2, 3 or 4 paws.

On the day of the test mice were placed on the glass runway and allowed to run freely back and forth. About 6 straight, consistent runs were recorded by the camera for each mouse. The 3 best runs with the closest total run time were selected for each mouse for analysis. For these three runs, the paw prints were labelled in successive frames using the software, which then analyzed multiple gait parameters for each of the runs. Using Excel, the average of all three runs per mouse was calculated, followed by the average per group for each of the gait parameters.

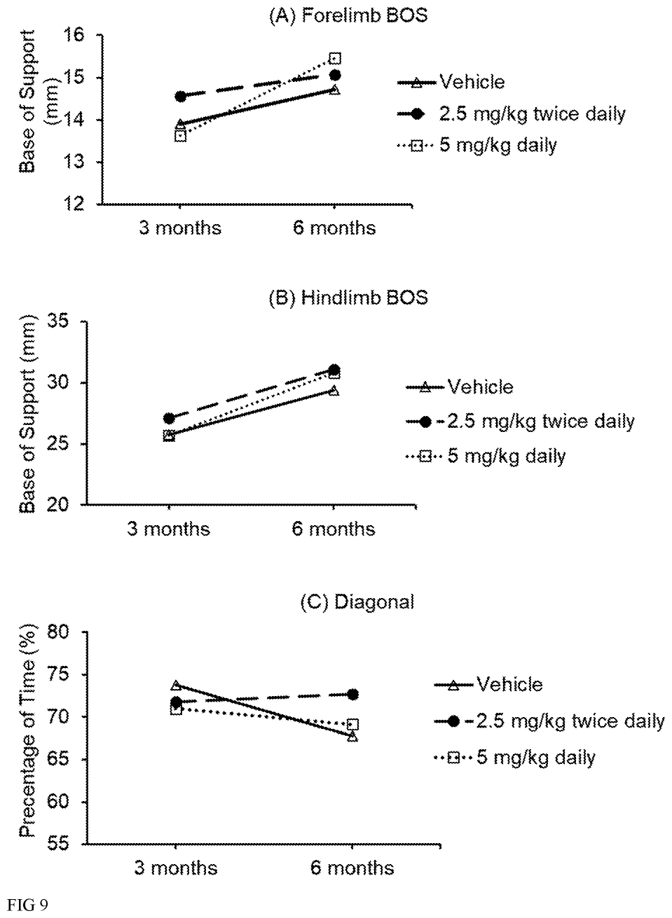

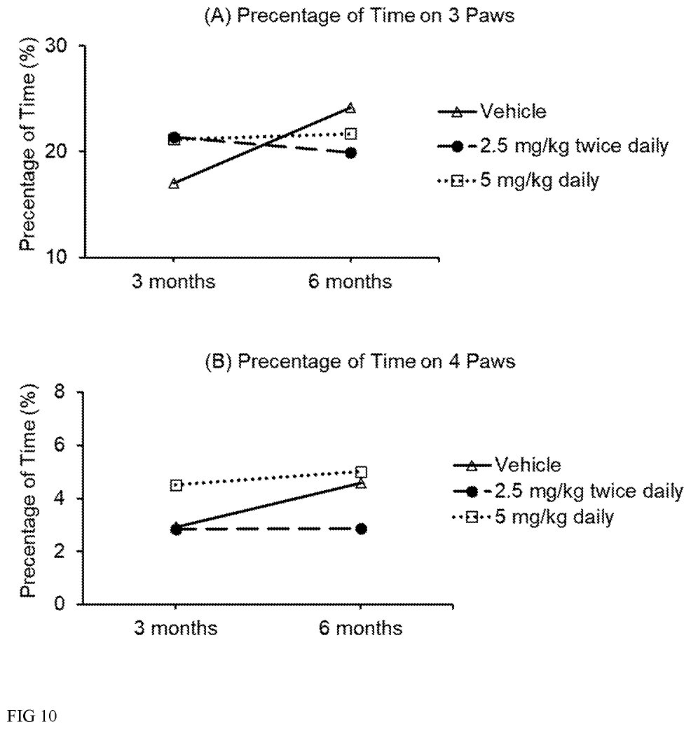

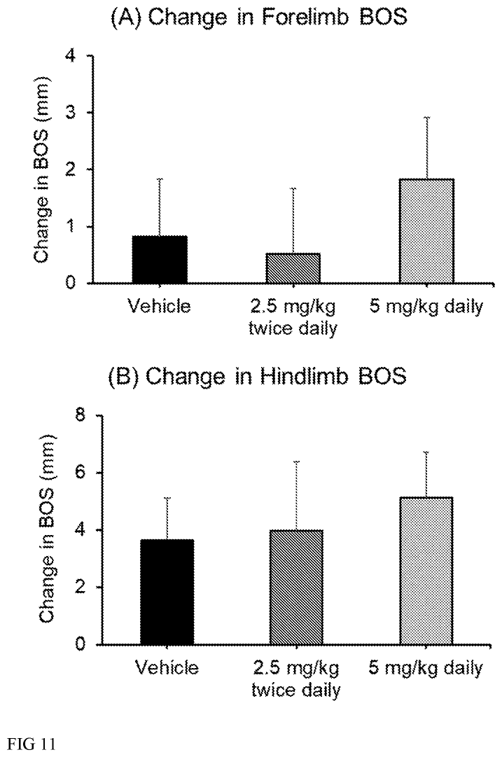

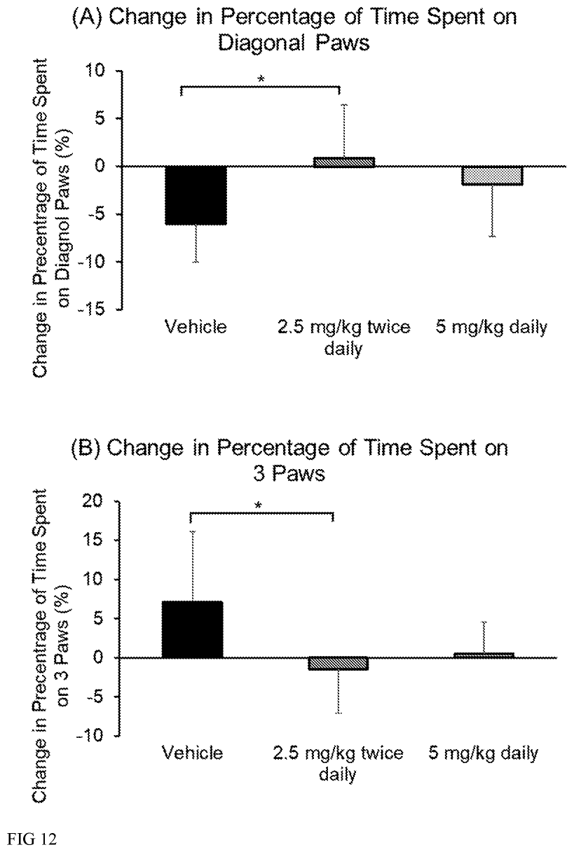

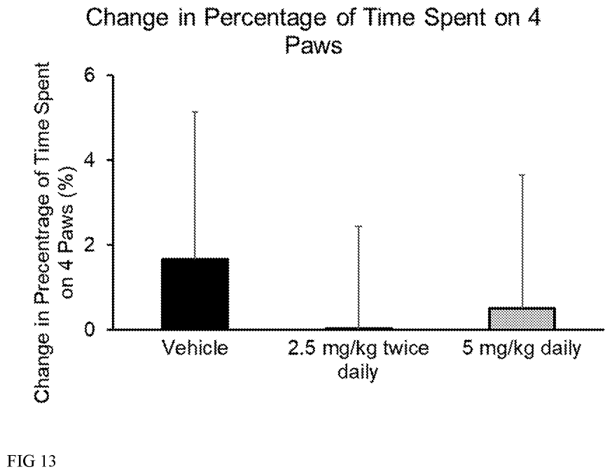

Gait analysis was performed using the catwalk system (Noldus) at both 3 and 6 months of age in 8 mice per group per timepoint. Traditionally in this model, the base of support increases with age, as shown in FIG. 9-13, represented the waddling or ‘swimming’ gait described in this strain. (6aS)-6-methyl-5,6,6a,7-tetrahydro-4H-dibenzo[de,g]quinoline-10,11-diol did not limit this swimming gait. The vehicle treated mice showed a decrease in the amount of time spent on diagonal paws and an increase in the percentage of time spent on three or four paws, indicating unsteady gait. The opposite is true for both the 2.5 and 5 mg/kg dosing groups, where these gait parameters are relatively stable from 3 to 6 months.

Electrophysiology (CMAP and Repetitive Stimulation)

Electrophysiological assessment of compound action muscle potential (CMAP) and repetitive stimulation was carried out at 6 weeks, 3 months and 6 months of age.

Mice were anesthetized using gaseous isoflurane and then maintained under gaseous anesthesia using a nose cone for the duration of the experiment. Body temperature was maintained with a heat pad. The fur from the lower left limb was removed using an electric razor followed by hair removal cream in order to allow skin contact of the ring electrodes. Ring electrodes were covered in electrical paste and were placed around the ankle and thigh area of the shaved limb. The rings were tightened so that there were no air gaps between the skin and electrodes but not so tight that blood flow was altered. A grounding electrode was placed into the base of the tail and stimulating electrodes were placed higher on the leg as near as possible to the sciatic nerve.

CMAPs were acquired by pulsing an electrical wave of 0. ms duration to the sciatic notch. The position of the electrodes was tested prior to the final pulse in order to make sure they were placed correctly by visualizing the outcome of the pulse. The stimulation current was then increased until no further increase in CMAP was seen.

Repetitive stimulation was carried out after the CMAP was calculated. The electrodes were held stable while ten pulses at 10 Hz were sent through the stimulating electrodes. Each of the 10 stimulations generated an amplitude and area for that stimulation. The data was normalized so that the first stimulation was 100%.

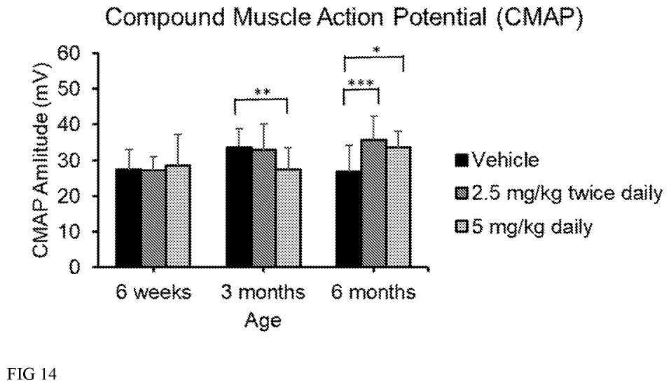

At 6 weeks of age, there is no significant difference between the different dosing groups when comparing CMAP. This average is similar to previous experiments in this disease model at this age. At 3 months of age there is a significant decrease in the 5 mg/kg (6aS)-6-methyl-5,6,6a,7-tetrahydro-4H-dibenzo[de,g]quinoline-10,11-diol dosing group when compared to the vehicle dosing group. At 6 months of age there is a significant increase in both the (6aS)-6-methyl-5,6,6a,7-tetrahydro-4H-dibenzo[de,g]quinoline-10,11-diol dosing groups when compared to vehicle dosed animals (FIG. 14).

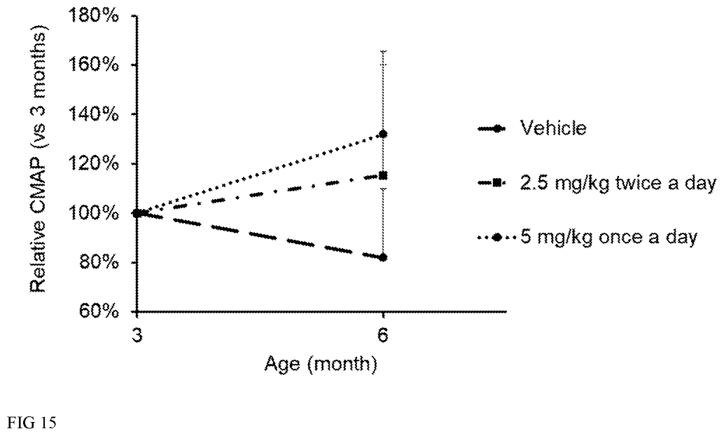

Given the variation among different subjects, the relative CMAP values at 6 months were also calculated based on the CMAP values of individual animals (FIG. 15). Both 2.5 mg/kg and 5 mg/kg dosing groups showed significant improvement of relative CMAP, compared to that of the vehicle group, indicating an improvement of electrophysiology by (6aS)-6-methyl-5,6,6a,7-tetrahydro-4H-dibenzo[de,g]quinoline-10,11-diol.

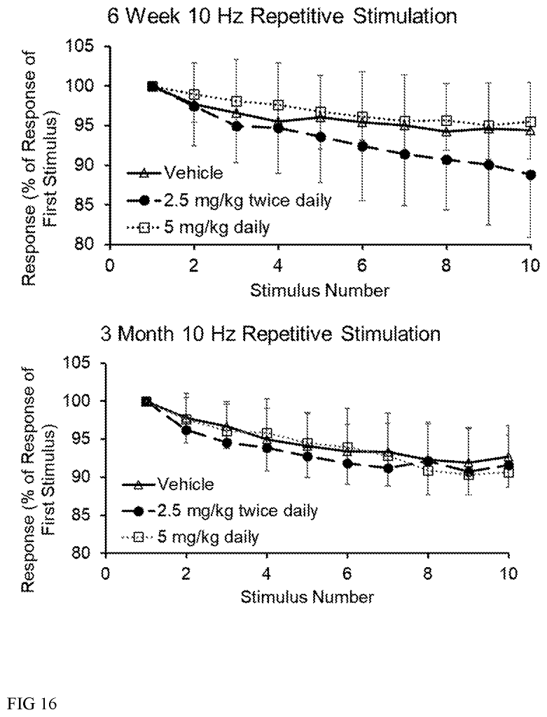

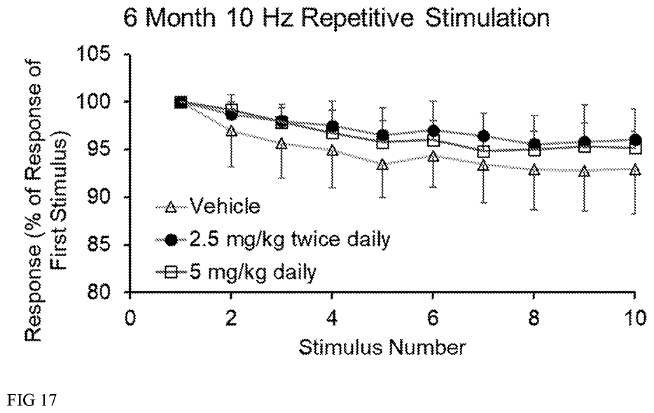

Repetitive stimulation is plotted as the percentage of the first stimulation to show decrease in response over multiple stimulations. In the TDP-43^{Q331K} model, a decrease in response over the 10 stimulations at 3 and 6 months of age was observed. At 6 weeks of age, there is a significant difference between the 2.5 mg/kg twice daily (6aS)-6-methyl-5,6,6a,7-tetrahydro-4H-dibenzo[de,g]quinoline-10,11-diol dosing group when compared to the vehicle dosed mice at the final stimulation, where (6aS)-6-methyl-5,6,6a,7-tetrahydro-4H-dibenzo[de,g]quinoline-10,11-diol dosed mice have a larger decrease in response from the first stimuli. The repetitive stimulation is difficult at 6 weeks due to the small size of the mice and their muscles at this age and so this may account for this difference as normally you would not see a difference so early in the disease model. There is no significant difference in repetitive stimulation between the dose groups at 3 months and there is a larger decrease in response over the stimulations at 3 months when compared to 6 weeks. There is a significant increase in response of the 2.5 mg/kg (6aS)-6-methyl-5,6,6a,7-tetrahydro-4H-dibenzo[de,g]quinoline-10,11-diol dose group when compared to the vehicle dosing group at 6 months of age at stimulation numbers 5 and 7 (FIGS. 16 and 17).

Tissue Collection

Tissue was collected at 24 hours after the final morning dose. All animals were overdosed with an intraperitoneal injection of pentibarital (JML, M042) at the time of tissue collection (2.5 ml/kg). In the 6 month cohort (n=14), 7 animals per group were perfused to fix tissue for histology. For these animals, once animals had lost pedal reflex, the chest cavity was opened and 10 ml of PBS was perfused via the heart, followed by 10 ml of 4% PFA. Brain and spinal cord were extracted and stored in 4% PFA overnight before being changed to PBS. The lumbar section of the spinal cord was dissected and embedded. For the remaining 7 animals per group, once the animals had lost pedal reflex, blood was extracted via a cardiac puncture technique. The blood was collected and stored in 1 ml of RNALater (ThermoFisher). Spinal cords were removed and the upper section of spinal cord was snap frozen in liquid nitrogen for protein analysis, while the lower section was stored in RNALater and stored at −20° C. Cortex was removed and dissected into four parts, the front left segment was stored in RNALater, while the three other sections were snap frozen in liquid nitrogen.

For the 3 month cohort, tissue was collected in a similar way to the snap frozen tissue for 6 month cohort, however where tissue was stored in RNALater for the 6 month cohort, it was processed for RNA extraction immediately in the 3 month cohort.

RNA Extraction

RNA was extracted from lower spinal cord and cortex using the RNeasy lipid tissue mini kit (Qiagen, 74804) following the manufactures protocol. Briefly tissue was homogenized in a small volume (150 μl) of QIAzol using a hand-held homogenizer in a fume hood. Once homogenized, the total volume of QIAzol was increased to 1 ml. Samples were incubated at room temperature for 5 minutes after which 200 μl of chloroform (Honeywell) was added and samples were shaken vigorously for 15 seconds. Mixed samples were then incubated at room temperature for 3 minutes then centrifuged at 12,000g for 15 minutes at 4° C. in a bench top microcentrifuge.

The upper aqueous phase of the sample was transferred to a fresh labelled tube and 1 volume of 70% ethanol was added. Samples were vortexed and 700 μl of sample was transferred to RNeasy mini spin columns attached to 2 ml collection tubes. The samples were centrifuged at room temperature for 15 seconds at 13,000 rpm and the flow-through was discarded. The remainder of the sample was added to the spin columns, the samples were spun again and flow-through was discarded. A 700 μl volume of RW1 buffer was added to the spin columns and these were spun at 13,000 rpm for 15 seconds. Flow-through was discarded. A 500 μl of RPE buffer was added to the columns, which were spun and flow through was discarded. A further 500 μl of RPE buffer was added to the column and the samples were centrifuged for 2 minutes at 13,000 rpm. To further dry the membrane, the spin column was placed into a fresh 2 ml tube and centrifuged at full speed for 1 minute. Finally, the column was placed in a fresh 1.5 ml tube and 30 μl of RNase-free water was added. These were centrifuged for 1 minute at 13,000 rpm and the flow-through was kept.

Quantification of RNA

Directly after extraction, RNA was quantified and checked for purity using a nanodrop ND-1000 (Thermo Scientific) by spectrophotometry. The total RNA concentration as well as A260/280 and A260/230 ratios were determined to check for purity of the sample.

cDNA Synthesis

All water used in the synthesis of cDNA and throughout the qPCR protocol was DEPC treated water. This was created by adding 1 ml of DEPC (BioChemica) to 1 L of MQ water and autoclaving.

cDNA was synthesized from the RNA using the following method. Firstly, any potential DNA was digested from the samples using RNase-free DNase and DNase buffer (Roch Diagnostics, 04716728001). A 1 μl volume of DNase and 1 μl of 10× DNase buffer was added to 2000 ng of RNA sample (total volume of 10 μl). This was then incubated at 37° C. for 10 minutes. The DNase was inactivated using 1 μl of 25 mM sterile DEPC treated EDTA (Amresco) and incubating at 75° C. for 10 minutes.

A 1 μl volume of DN6 (random hexamer primers, Sigma Aldrich) and 1 μl deoxyribonucleotide triphosphates (dNTP, bioline, BIO-39053) were added to each reaction and these were incubated at 75° C. for 5 minutes to denature the RNA. Samples were placed on ice immediately to prevent refolding of RNA and 2 μl of DTT, 4 μl 5× buffer and 1 μl reverse transcriptase (RT) enzyme was added to all tubes (all Invitrogen, 28025-013). These were placed into a PCR machine (G-storm) and run on the following protocol: 25° C. for 10 minutes, 42° C. for 1 hour, 85° C. for 5 minutes then hold at 10° C. Once the protocol has finished, 40 μl of DEPC H₂O was added, samples were briefly vortexed and cDNA was stored at −20° C.

qPCR

Primers (Sigma Aldrich) were diluted to 100 μM using DEPC H₂O. rimers were further diluted to create primer mixes that contained both the forward and reverse primers at concentrations optimized for each target.

All qPCR experiments were carried out using 96 well qPCR plates (Bio-Rad, MLL9651) with optical strip lids (Bio-Rad, TCS0803) or 384 well plates (Bio-Rad, HSP3865) with clear plate seals.

Cycle threshold (cT) values, amplification curves and melt peaks were analyzed and extracted using CFX Maestro software (Bio-Rad) and further analyzed using Excel (Microsoft) and GraphPad Prism 7. Relative mRNA levels were detected by normalizing to an endogenous control and normalization to vehicle samples using the ΔΔCT method.

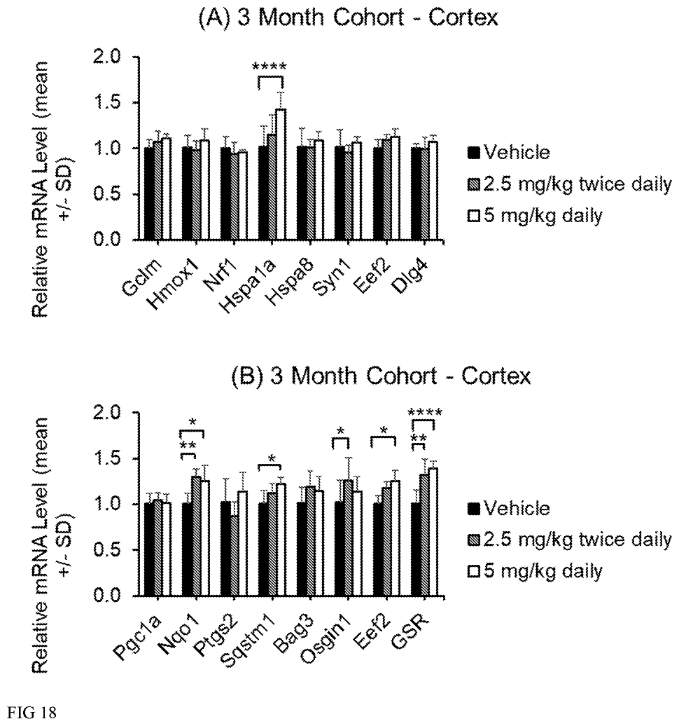

At 3 months, cortex samples from mice (normalized to Gapdh, n=6) showed a significant upregulation for Hspa1a, Nqo1, Sqstm1 and GSR at 5 mg/kg dosing group, compared to that of the vehicle group. An upregulation of Nqo1, Osgin1 and GSR was also observed at 2.5 mg/kg dosing group (FIG. 18).

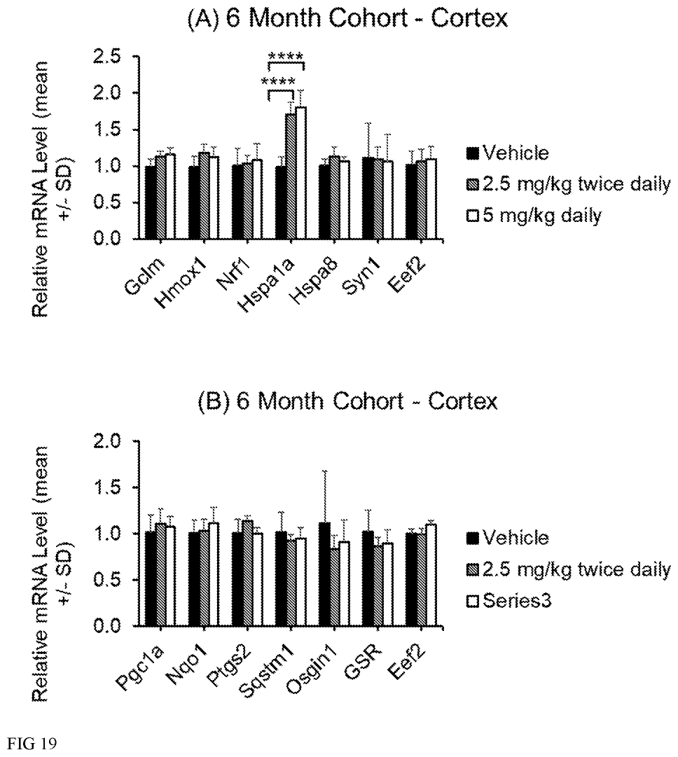

At 6 months, cortex samples from mice (normalized to Gapdh, n=7) showed a significant upregulation of Hspa1a at the dose level of 2.5 mg/kg twice daily and 5 mg/kg daily (FIG. 19).

### Example 5: Protein Analysis in the In Vitro Pharmacology Study

Over the last decade, in vitro modelling of neurodegeneration has undergone impressive development, mainly due to the reprogramming of adult human fibroblasts into induced pluripotent stem cells (iPSCs) and induced neural progenitor cells (iNPCs). In the ALS research field, this offers an opportunity to model familial and sporadic diseases in vitro.

NPCs harvested from post mortem spinal cord of ALS patients have already been successfully differentiated into motor neurons, astrocytes and oligodendrocytes. Deriving astrocytes using this method avoids inducing major epigenetic alterations. However, the availability of post-mortem samples is limited. In addition, the disadvantages of reprogramming astrocytes from human derived iPSCs include time-consuming protocols, as well as complex and highly-variable maturation time of the astrocytes.

Therefore, a promising alterative to iPSC resources is the direct reprogramming of fibroblasts into astrocytes from an immuno-matched host. Instead of generating iPSCs, direct reprogramming involves the use of cell-lineage transcription factors to convert adult somatic cells into another cell type. This technology has been used to generate sub-specific neural lineages such as cholinergic, dopaminergic and motor neurons. Direct reprogramming technology was also used to derive astrocytes from ALS patient fibroblasts, and tripotent iNPCs from ALS patients and controls were generated within one month. When these cells were differentiated into astrocytes, they displayed similar toxicity towards motor neurons in co-cultures as autopsy-derived astrocytes, making them useful tools in the development of drug screens.

Methodology

Induced NPCs were generated from adult human fibroblasts from patients who had been diagnosed with ALS and from age-matched healthy controls, using an approach reported previously (Kim et al PNAS, 2001. 108(19), 7838-7843; Meyer et al., PNAS, 2014. 111(2), 829-832). Induced NPCs are differentiated into induced astrocytes (iAstrocytes) by culturing the progenitors in iAstrocyte medium for a total of 7 days with a medium change at day 3.

Induced astrocytes derived from human donors were treated with 0.1% DMSO, 10 uM of (6aS)-6-methyl-5,6,6a,7-tetrahydro-4H-dibenzo[de,g]quinoline-10,11-diol or 10 uM of Riluzole 48 hours before collection. Cells were scraped from 10 cm dishes and cell pellets were lysed in ice cold IP lysis buffer (150 mM NaCl, 50 mM HEPES, 1 mM EDTA, 1 mM DTT, 0.5% (v/v) Triton X-100, protease inhibitor cocktail, pH 8.0) for 15 minutes and further homogenized using a 25-gauge needle and syringe. Protein samples were separated by SDS-Polyacrylamide Gel Electrophoresis and then semi-dry transferred onto nitrocellulose membranes. Membranes were blotted with Anti-NQO1—1:1000 (5% milk/TBST); rabbit; abcam; ab34173 at 4° C. overnight and anti-Beta-actin—1:5,000 (5% milk/TBST); mouse; abcam; ab6276 clone AC-15 at 4° C. overnight.

Western Blot Analysis

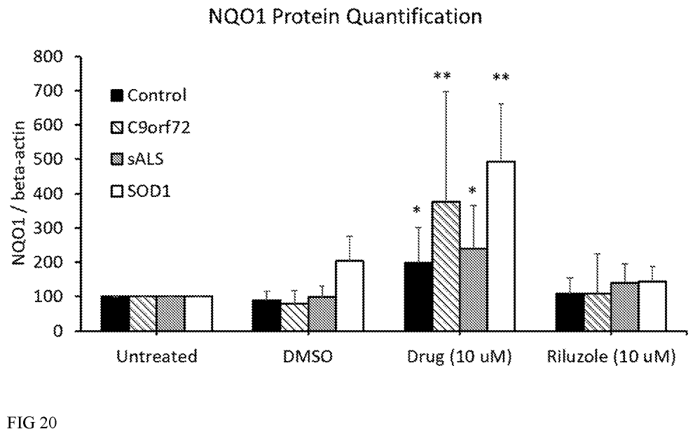

Protein quantification data from western blot analysis demonstrated that (6aS)-6-methyl-5,6,6a,7-tetrahydro-4H-dibenzo[de,g]quinoline-10,11-diol induced a significant increase in NQO1 after 48 hours treatment at 10 uM in human iAstrocytes. As shown in FIG. 20, human iAstrocytes were derived from healthy individuals (CTR, n=3), patients carrying C9orf72 mutations (C9orf72, n=3); sporadic ALS patients (sALS, n=3) and patients carrying SOD1 mutations (SOD1, n=3).

1-74. (canceled)

75. A method of treating a disease or disorder in a subject in need thereof, the method comprising the step of administering a therapeutically effective amount of a pharmaceutical composition comprising 6-methyl-5,6,6a,7-tetrahydro-4H-dibenzo[de,g]quinoline-10,11-diol to the subject.

76. The method of claim 75, wherein the pharmaceutical composition comprises a racemic mixture of (6aR)-6-methyl-5,6,6a,7-tetrahydro-4H-dibenzo[de,g]quinoline-10,11-diol and (6aS)-6-methyl-5,6,6a,7-tetrahydro-4H-dibenzo[de,g]quinoline-10,11-diol.

77. The method of claim 75, wherein the pharmaceutical composition comprises (6aS)-6-methyl-5,6,6a,7-tetrahydro-4H-dibenzo[de,g]quinoline-10,11-diol.

78. The method of claim 75, wherein the disease or disorder is aging-related tau astrogliopathy (ARTA), Alexander Disease, Alpers-Huttenlocher syndrome, Alzheimer's disease, Amyotrophic Lateral Sclerosis (ALS), Ataxia neuropathy spectrum, ataxia and retinitis pigmentosa (NARP), critical illness myopathy (CIM), primary age-related tauopathy (PART), aortic medial amyloidosis, ApoAI amyloidosis, ApoAII amyloidosis, ApoAIV amyloidosis, argyrophillic grain disease, ataxia telangiectasia, atrial fibrillation, autosomal dominant hyper-IgE syndrome, cardiac atrial amyloidosis, Bloom's syndrome, cardiovascular diseases, coronary artery disease, myocardial infarction, stroke, restenosis, arteriosclerosis, cataracts, cerebral amyloid angiopathy, Christianson syndrome, chronic traumatic encephalopathy, chronic progressive external opthalmoplegia (CPEO), Cockayne's syndrome, congenital lactic acidosis (CLA), corneal lactoferrin amyloidosis, corticobasal degeneration, Crohn's disease, Cushing's disease, cutaneous lichen amyloidosis, cystic fibrosis, Dentatorubropallidoluysian Atrophy (DRPLA), dialysis amyloidosis, diffuse neurofibrillary tangles with calcification, Down syndrome, endotoxin shock, familial amyloidosis of the Finnish type, familial amyloidotic neuropathy, familial British dementia (FBD), familial Danish dementia (FDD), familial dementia, fibrinogen amyloidosis, fragile X syndrome, fragile X-associated tremor/ataxia syndrome (FXTAS), Friedreich's ataxia, fronto-temporal degeneration, glaucoma, glycogen storage disease type IV (Andersen disease), Guadeloupean Parkinsonism, hereditary lattice corneal dystrophy, Huntington's disease, inclusion body myositis/myopathy, inflammation, inflammatory bowel disease, ischemic condition, ischemia/reperfusion injury, myocardial ischemia, stable angina, unstable angina, stroke, ischemic heart disease and cerebral ischemia, light chain or heavy chain amyloidosis, lysosomal storage diseases, aspartylglucosaminuria, Fabry's disease, Batten disease, cystinosis, Farber, fucosidosis, galactasidosialidosis, Gaucher's disease Type 1, 2 or 3, Gml gangliosidosis, Hunter's disease, Hurler-Scheie's disease, Krabbe's disease, a-mannosidosis, Kearns-Sayre syndrome (KSS), lactic acidosis and stroke-like episodes (MELAS) syndrome, Leber hereditary optic neuropathy (LHON), B-mannosidosis, Maroteaux-Lamy's disease, MEGDEL syndrome (also known as 3-methylglutaconic aciduria with deafness, encephalopathy and Leigh-like syndrome), metachromatic leukodystrophy, mitochondrial neurogastro-intestinal encephalopathy (MNGIE) syndrome, Morquio A syndrome, Morquio B syndrome, mucolipidosis II, mucolipidosis III, myoclonic epilepsy myopathy sensory ataxia, mitochondrial myopathy, myoclonic epilepsy with ragged red fibres (MERRF), Neimann-Pick Disease Type A, B or C, neurogenic muscle weakness, Pearson syndrome, Pompe's disease, Sandhoff disease, Sanfilippo syndrome Type A, B, C or D, Schindler disease, Schindler-Kanzaki disease, Sengers syndrome, sialidosis, Sly syndrome, Tay-Sach's disease, Wolman disease, lysozyme amyloidosis, mallory bodies, medullary thyroid carcinoma, mitochondrial myopathies, multiple sclerosis, multiple system atrophy, myotonic dystrophy, myotonic dystrophy, neurodegeneration with brain iron accumulation, neurofibromatosis, neuronal ceroid lipofuscinosis, odontogenic (Pinborg) tumor amyloid, Parkinsonism-Dementia of Guam, Parkinson's disease, peptic ulcers, Pick's disease, pituitary prolactinoma, post encephalitic Parkinsonism, prion diseases (transmissible spongiform encephalopathies), including Creutzfeldt-Jakob disease (CJD), variant Creutzfeldt-Jakob disease, Gerstmann-Straussler-Scheinker Syndrome, fatal familial insomnia, Kuru, progressive supranuclear palsy, pulmonary alveolar proteinosis, retinal ganglion cell degeneration in glaucoma, retinitis pigmentosa with rhodopsin mutations, seminal vesical amyloid, senile systemic amyloidoses, serpinopathies, sickle cell disease, spinal and bulbar muscular atrophy (SBMA), spinocerebellar ataxias, spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 8, spinocerebellar ataxia type 17), subacute sclerosing panencephalitis, tauopathies, type II diabetes, vascular dementia, Werner syndrome, atherosclerosis, autism spectrum disorder (ASD), benign focal amyotrophy, Duchenne's paralysis, hereditary spastic paraplegia (HSP), Kugelberg-Welander syndrome, Lou Gehrig's disease, necrotizing enterocolitis, Paget's disease of the bone (PDB), primary lateral sclerosis (PLS), progressive bulbar palsy (PBP), progressive muscular atrophy (PMA), pseudobulbar palsy, spinal muscular atrophy (SMA), ulcerative colitis, valosin-containing protein (VCP)-related disorders, or Werdnig-Hoffmann disease, transient ischemic attack, ischemia, cerebral hemorrhage, senile cataract, retinal ischemia, retinal vasculitis, Brown-Vialetto-Van Laere syndrome, Eales disease, meningitis and encephalitis, post-traumatic stress disorder, Charcot-Marie-Tooth disease, macular degeneration, X-Linked bulbo-spinal atrophy, presenile dementia, depressive disorder, temporal lobe epilepsy, hereditary Leber optic atrophy, cerebrovascular accident, subarachnoid hemorrhage, schizophrenia, demyelinating disorders, or Pelizaeus-Merzbacher disease.

79. The method of claim 75, wherein the disease or disorder is a neurodegenerative disease or disorder.

80. The method of claim 79, wherein the disease or disorder is ALS, frontotemporal dementia, Huntington's disease, Alzheimer's disease, Parkinson's disease, dementia with Lewy bodies, Parkinson's disease dementia, vascular dementia, Creutzfeldt-Jakob disease, fatal familial insomnia, Gerstmann-Straussler-Scheinker syndrome, kuru, or ataxia.

81. The method of claim 80, wherein the disease or disorder is ALS.

82. The method of claim 80, wherein the disease or disorder is Parkinson's disease.

83. The method of claim 80, wherein the disease or disorder is Huntington's disease,

84. The method of claim 80, wherein the disease or disorder is Friedreich's ataxia.

85. The method of claim 75, wherein the therapeutically effective dose is at least 0.12 mg/kg.

86. The method of claim 75, wherein the therapeutically effective dose is between 5 mg/day and 5000 mg/day.

87. The method of claim 75, wherein the 6-methyl-5,6,6a,7-tetrahydro-4H-dibenzo[de,g]quinoline-10,11-diol is administered by oral administration.

88. A pharmaceutical composition comprising (6aS)-6-methyl-5,6,6a,7-tetrahydro-4H-dibenzo[de,g]quinoline-10,11-diol and at least one pharmaceutically acceptable excipient.

89. The pharmaceutical composition of claim 88, wherein the pharmaceutical composition is formulated for oral administration.

90. The pharmaceutical composition of claim 88, wherein the pharmaceutical composition is formulated for subcutaneous administration.


SEQUENCE LISTING (9 sequences)

SEQ ID NO: 1 - DNA, 20 bp, Artificial Sequence
  Mus-Actb-taqman-F primer
tggaatcctgtggcatccat

SEQ ID NO: 2 - DNA, 20 bp, Artificial Sequence
  Mus-Actb-taqman-R primer
gctaggagccagagcagtaa

SEQ ID NO: 3 - DNA, 25 bp, Artificial Sequence
  Mus-Actb-probe
accaccagacagcactgtgttggca

SEQ ID NO: 4 - DNA, 20 bp, Artificial Sequence
  Mus-Hspa1a-taqman-F primer
gctgcttctccttgcgttta

SEQ ID NO: 5 - DNA, 20 bp, Artificial Sequence
  Mus-Hspa1a-taqman-R primer
tgctgtcacttcacctccaa

SEQ ID NO: 6 - DNA, 25 bp, Artificial Sequence
  Mus-Hspa1a-probe
agtcctacagtgcaaccaccatgca

SEQ ID NO: 7 - DNA, 20 bp, Artificial Sequence
  Mus-Hspa8-taqman-F primer
tggaactattgctggcctca

SEQ ID NO: 8 - DNA, 20 bp, Artificial Sequence
  Mus-Hspa8-taqman-R primer
ttcctttcagctccgacctt

SEQ ID NO: 9 - DNA, 24 bp, Artificial Sequence
  Mus-Hspa8-probe
actgctgctgctattgcttacggc